(19) United States
(12) Patent Application Publication
(10) Pub. No.: US 2026/0234096 A1
(43) Pub. Date: Aug. 13, 2026

(54) CATALYST FOR SP3-CARBON-HYDROGEN-BOND PRIMARY AMINATION REACTION, AND METHOD FOR PREPARING PRIMARY AMINE COMPOUND

(71) Applicant: WEST CHINA SECOND UNIVERSITY HOSPITAL, SICHUAN UNIVERSITY, Chengdu, Sichuan (CN)

(72) Inventors: Zhijun JIA, Chengdu, Sichuan (CN); Ye LIU, Chengdu, Sichuan (CN); Peng CHEN, Chengdu, Sichuan (CN)

(21) Appl. No.: 19/154,102

(22) PCT Filed: Feb. 5, 2024
(86) PCT No.: PCT/CN2024/075973

§ 371 Date: Aug. 6, 2025

(30) Foreign Application Priority Data
Feb. 8, 2023 (CN) 202310108912.X

(51) Int. Cl. C07C 209/02; B01J 31/18; B01J 31/22; C07C 213/02; C07C 221/00; C07C 227/06; C07C 253/30; C07D 207/06; C07D 207/26; C07D 207/267; C07D 209/46; C07D 211/12; C07D 211/76; C07D 215/44; C07D 307/81; C07D 307/82; C07D 311/68; C07D 311/72; C07D 333/20; C07J 41/00

(52) U.S. Cl. CPC C07C 209/02; B01J 31/183; B01J 31/2295; C07C 213/02; C07C 221/00; C07C 227/06; C07C 253/30; C07D 207/06; C07D 207/26; C07D 207/267; C07D 209/46; C07D 211/12; C07D 211/76; C07D 215/44; C07D 307/81; C07D 307/82; C07D 311/68; C07D 311/72; C07D 333/20; C07J 41/0011; B01J 2231/44; B01J 2531/004; B01J 2531/0213; B01J 2531/0258; B01J 2531/842; C07C 2601/02; C07C 2601/08; C07C 2601/14; C07C 2601/16; C07C 2601/18; C07C 2601/20; C07C 2602/06; C07C 2602/08; C07C 2602/10; C07C 2602/12; C07C 2602/28; C07C 2602/42; C07C 2603/18; C07C 2603/20; C07C 2603/26; C07C 2603/74; C07C 2603/78

(57) ABSTRACT

The present invention belongs to the technical field of chemical synthesis. Provided are a catalyst for an sp³-carbon-hydrogen-bond primary amination reaction, and a method for preparing a primary amine compound. The structure of the catalyst is as represented by formula (I). The method for preparing a primary amine compound by using the catalyst in the present invention has high general applicability and wide substrate applicability for reaction, is environmentally friendly; the reaction conditions are mild and not harsh, and the reaction operation is simple and convenient; and the method has potential industrial application prospects.

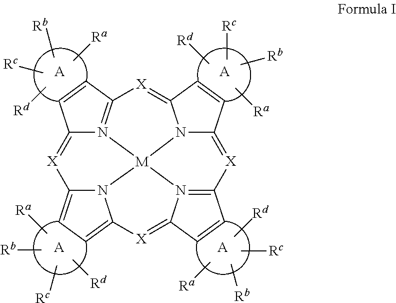

### FIELD OF THE INVENTION

The present invention belongs to the technical field of chemical synthesis, and specifically relates to a catalyst for the primary amination reaction of sp³-carbon-hydrogen bonds and a method for preparing primary amine compounds.

### BACKGROUND OF THE INVENTION

The aliphatic primary amine structure is not only widely present in bioactive molecules (such as amoxicillin, sitagliptin/metformin extended-release tablets Janumet, memantine, etc.), but also serves as an important intermediate for chemical synthesis. It is widely used in the synthesis of natural products, drug molecules, and agricultural chemicals. Furthermore, primary amines can also act as the important precursors for further constructing secondary amines, tertiary amines, and heterocyclic structures.

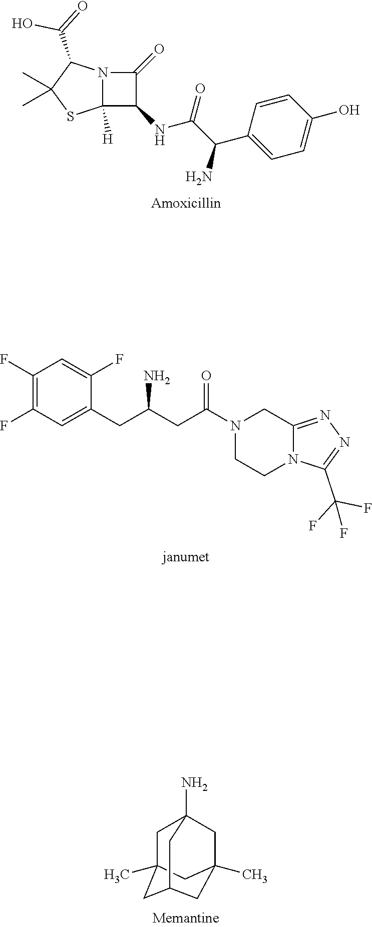

Traditional methods for synthesizing primary amines primarily rely on organic transformation reactions involving special functional groups, including the Gabriel reaction, Leuckart reaction, Curtius rearrangement reaction, Schmidt reaction, and reduction reactions of imino, azido, nitrile, and nitro groups. However, these methods not only require the introduction of pre-functional groups into the raw materials in advance, resulting in low atom economy, but also have the disadvantages of harsh reaction conditions, numerous by-products, and environmental unfriendliness. Therefore, developing an efficient method for synthesizing primary amines from simple and readily available raw materials holds great application value.

sp³-carbon-hydrogen bonds are widely present in natural products and bulk chemical raw materials, with the advantages of being widely sourced and easily accessible. Therefore, the direct synthesis of primary amines from sp³-carbon-hydrogen bonds exhibits great atom economy and applicability. However, chemical synthesis methods currently reported can only prepare amines with protective groups from sp³-carbon-hydrogen bonds, which require further deprotection to obtain the corresponding primary amines. These methods have some obvious drawbacks, such as the use of precious metals (rhodium, iridium), harsh reaction conditions (such as high temperature, inert gas protection, and the use of highly toxic organic solvents (like benzene), etc.). So far, no chemical method has been reported for directly synthesizing primary amines from sp³-carbon-hydrogen bonds under mild conditions using economically accessible and environmentally friendly catalysts.

From 2020 to 2022, F. H. Arnold team continuously developed methods for synthesizing primary amines by enzymatic catalysis of sp³-carbon-hydrogen bonds. By simulating the process of biological oxidation catalyzed by P450 enzymes on sp³-carbon-hydrogen bonds, they designed a nitrene intermediate that can be formed between the iron catalytic center in P450 enzymes and the PivONH₃OTf amine source, thus achieving primary amination of sp³-carbon-hydrogen bonds. By directed evolution of cytochrome P450 enzymes, a library of different P450 enzyme variants was obtained, enabling primary amination of sp³-carbon-hydrogen bonds in benzyl, allyl, propargyl, and alkane compounds. Moreover, these variants exhibited high selectivity and reactivity towards most substrates (J. Am. Chem. Soc. 2020, 142, 23, 10279-10283; J. Am. Chem. Soc. 2022, 144, 1, 80-85; J. Am. Chem. Soc. 2022, 144, 41, 19097-19105).

However, the above method still has the following shortcomings:
1. The reaction substrates are relatively limited, and especially for alkane substrates, the reaction activity and selectivity are poor. Most of the products cannot be obtained by separation and purification, rendering them of no practical value;
2. This method is not suitable for direct primary amination modification of complex natural products or active pharmaceutical molecules, nor has it been applied to the direct synthesis of some important drug molecules. Furthermore, it has not achieved for it to be used in the tandem cyclization reactions for the efficient construction of cyclic secondary amines and lactam compounds;
3. This method requires stringent reaction conditions, which must be carried out under strict anaerobic conditions, making it unsuitable for industrial scale-up applications.

Therefore, it is of great significance to further develop new primary amination processes for sp³-carbon-hydrogen bonds that are highly versatile and exhibit mild reaction conditions.

### Content of the Invention

The objective of the present invention is to provide a catalyst for the primary amination reaction of sp³-carbon-hydrogen bonds and a method for preparing primary amine compounds.

The present invention provides the use of the compound represented by formula I as a catalyst for primary amination of sp³-carbon-hydrogen bonds:

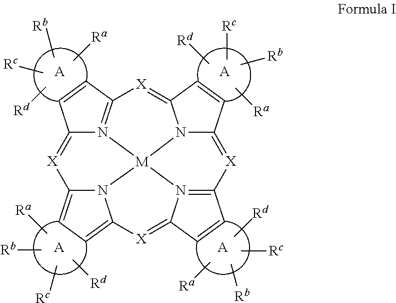

wherein, fused ring A is selected from the group consisting of

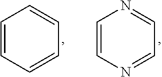

or absence; R^{a}, R^{b}, R^{c}, and R^{d} are each independently selected from the group consisting of H, halogen, alkyl, alkoxy, nitro, carboxylic acid, ester group, and alternatively, any two of R^{a}, R^{b}, R^{c}, and R^{d} are linked to form a ring; X is N or CR^{e}, and R^{e} is selected from the group consisting of aromatic rings or substituted aromatic rings or aromatic heterocycles with any group; M is a metal ion.

Further, R^{a}, R^{b}, R^{c}, and R^{d} are each independently selected from the group consisting of H and halogens, and at least one of them is halogen.

Further, said M is selected from the group consisting of Fe²⁺, Fe³⁺, Co²⁺, Ni²⁺, or Mn²⁺. Further, said M is Fe²⁺.

Further, said fused ring A represents

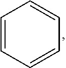

X represents nitrogen, and the halogen represents chlorine or fluorine.

Further, R^{a} and R^{d} are hydrogen, while R^{b} and R^{c} are chlorine.

Further, the compound represented by formula I is iron octachlorophthalocyanine.

Further, the primary amination of sp³-carbon-hydrogen bond involves the use of compound 1 and compound 2 as reactants, which are catalyzed by a catalyst to produce compound 3, and the reaction scheme is as follows:

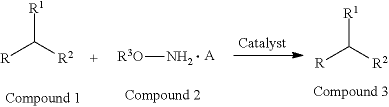

wherein, R, R¹, and R² are each independently selected from any groups, and alternatively, any two or three of R, R¹, and R² are linked to form a substituted or unsubstituted ring; R³ is selected from the group consisting of H, pivaloyl, acetyl, methanesulfonyl, p-toluenesulfonyl, sulfonic acid group, nitro, or methyl; A is selected from the group consisting of sulfonic acid, hydrochloric acid, sulfuric acid, acetic acid, or absence.

Further, R³ represents pivaloyl; A represents sulfonic acid, and preferably trifluoromethanesulfonic acid.

Further, the reaction conditions are: reacting in a solvent at 20-100° C. for 12-120 h;
and/or, the molar ratio of compound 1 to compound 2 is 1:(1-3), the molar ratio of compound 1 to the catalyst is 100:(2-10), and the concentration of compound 1 is 0.00625 M to 0.05 M.

Further, the reaction temperature is 25° C., and the reaction time is 12 h;
and/or, the molar ratio of compound 1 to compound 2 is 1:3, the molar ratio of compound 1 to the catalyst is 100:5, and the concentration of compound 1 is 0.0125 M.

Further, the solvent is selected from the group consisting of acetonitrile, dioxane, water, hexafluoroisopropanol, dichloromethane, N,N-dimethylformamide, and a mixture thereof.

Further, the solvent is a mixed solvent of dioxane and water.

The present invention provides a method for preparing primary amine compounds, which comprises the step of preparing primary amine compound 3 by reacting compound 1 and compound 2 as reactants under the action of a catalyst; the reaction scheme is as follows:

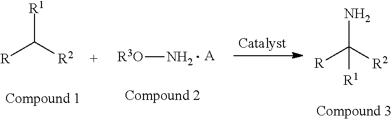

wherein, R, R¹, and R² are each independently selected from any groups, and alternatively, any two or three of R, R¹, and R² are linked to form a substituted or unsubstituted ring; R³ is selected from the group consisting of H, pivaloyl, acetyl, methanesulfonyl, p-toluenesulfonyl, sulfonic acid group, nitro, or methyl; A is selected from the group consisting of sulfonic acid, hydrochloric acid, sulfuric acid, acetic acid, or absence;
   the catalyst is a compound represented by formula I:

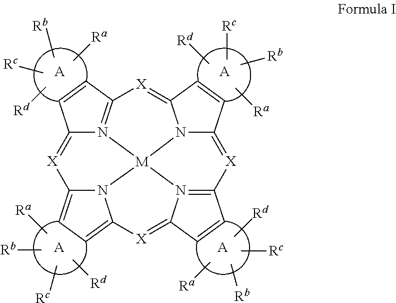

wherein, fused ring A is selected from the group consisting of

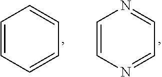

or absence; R^{a}, R^{b}, R^{c}, and R^{d} are each independently selected from the group consisting of H, halogen, alkyl, alkoxy, nitro, carboxylic acid, ester group, and alternatively, any two of R^{a}, R^{b}, R^{c}, and R^{d} are linked to form a ring; X is N or CR^{e}, and R^{e} is selected from the group consisting of aromatic rings or substituted aromatic rings or aromatic heterocycles with any group; M is a metal ion.

Further, R^{a}, R^{b}, R^{c}, and R^{d} are each independently selected from the group consisting of H and halogens, and at least one of them is halogen.

Further, said M is selected from the group consisting of Fe²⁺, Fe³⁺, Co²⁺, Ni²⁺, or Mn²⁺.

Further, said M is Fe²⁺.

Further, said fused ring A represents

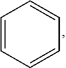

X represents nitrogen, and the halogen represents chlorine or fluorine.

Further, R^{a} and R^{d} are hydrogen, while R^{b} and R^{e} are chlorine.

Further, the compound represented by formula I is iron octachlorophthalocyanine.

Further, R³ represents pivaloyl; A represents sulfonic acid, and preferably trifluoromethanesulfonic acid.

Further, the reaction conditions are: reacting in a solvent at 20-100° C. for 12-120 h;
and/or, the molar ratio of compound 1 to compound 2 is 1:(1-3), the molar ratio of compound 1 to the catalyst is 100:(2-10), and the concentration of compound 1 is 0.00625 M to 0.05 M.

Further, the reaction temperature is 25° C., and the reaction time is 12 h;
and/or, the molar ratio of compound 1 to compound 2 is 1:3, the molar ratio of compound 1 to the catalyst is 100:5, and the concentration of compound 1 is 0.0125 M.

Further, the solvent is selected from the group consisting of acetonitrile, dioxane, water, hexafluoroisopropanol, dichloromethane, N,N-dimethylformamide, and a mixture thereof.

Further, the solvent is a mixed solvent of dioxane and water.

Further, R is a substituted or unsubstituted (5-6)-membered aromatic ring, (5-6)-membered aromatic heterocycle, (5-6)-membered fused (5-6)-membered aromatic ring, or (5-6)-membered fused (5-6)-membered aromatic heterocycle.

Further, R¹ and R² are each independently selected from any groups, and the reaction scheme is as follows:

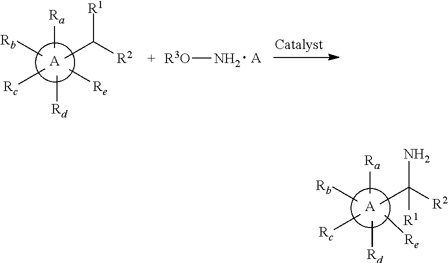

wherein, Rₐ, R_{b}, R_{c}, Rₐ, and Rₑ are each independently selected from H or any group other than H; ring A is selected from the group consisting of

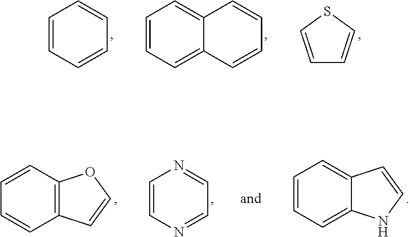

Further, said Rₐ, R_{b}, R_{c}, R_{d}, and Rₑ are each independently selected from the group consisting of H, halogen, phenyl substituted with straight or branched C₁₋₁₀ alkyl, phenyl, straight or branched C₁₋₁₀ alkyl, straight or branched C₁₋₁₀ alkoxy;
and/or, said R¹ and R² are each independently selected from H, (3-6)-membered saturated cycloalkyl, straight or branched C₁₋₁₀ alkyl, phenyl, phenyl-substituted C₁₋₁₀ straight or branched alkyl, or R₁ and R₂ are linked to form a ring.

Further, the reaction conditions are: reacting in a mixed solvent of dioxane and water at 20-80° C. for 12-120 h, preferably at 25-70° C. for 12-120 h, and more preferably at 25-60° C. for 12-120 h.

Further, R¹ is H, R² and R are linked to form a ring, and the reaction scheme is as follows:

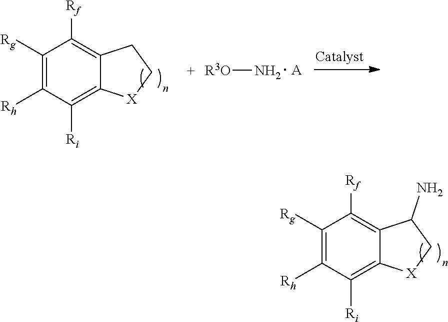

wherein, R_{f}, R_{g}, Rₕ, and Rᵢ are each independently selected from H or any group other than H; X is selected from the group consisting of CH₂, O, or NR′, and n is any integer from 0 to 6; R′ is selected from the group consisting of H, C₁₋₁₈ alkyl, benzyl, or amino-protecting group, and is preferably selected from the group consisting of H, methyl, ethyl, benzyl, benzoyl, or Boc.

Further, R_{f}, R_{g}, Rₕ, and Rᵢ are each independently selected from the group consisting of H, halogen, phenyl substituted with straight or branched C₁₋₁₀ alkyl, phenyl, straight or branched C₁₋₁₀ alkyl, straight or branched C₁₋₁₀ alkoxy; alternatively, two adjacent groups of R_{f}, R_{g}, Rₕ, and Rᵢ are linked to form a ring.

Further, the reaction conditions are: reacting in a mixed solvent of dioxane and water at 25-60° C. for 12-120 h.

Further, R represents substituted or unsubstituted alkenyl.

Further, R is

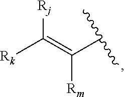

wherein Rⱼ, Rₖ, and Rₘ are any group.

Further, R¹ and R² are each independently selected from any group, and the reaction scheme is as follows:

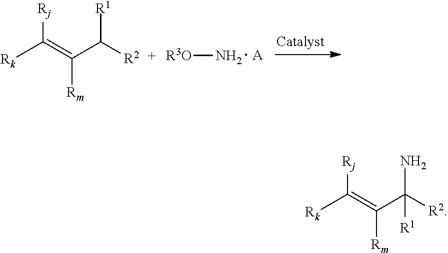

Further, R¹ and R² are each independently selected from the group consisting of H and straight or branched C₁₋₁₀ alkyl; Rⱼ, Rₖ, and Rₘ are each independently selected from the group consisting of H and straight or branched C₁₋₁₀ alkyl.

Further, the reaction conditions are: reacting in dioxane and water at 20-60° C. for 12-48 h, and preferably reacting at 25-40° C. for 12-24 h.

Further, said R is

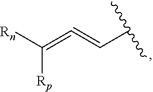

wherein Rₙ and Rₚ are any group.

Further, R¹ and R² are each independently selected from any group, and the reaction scheme is as follows:

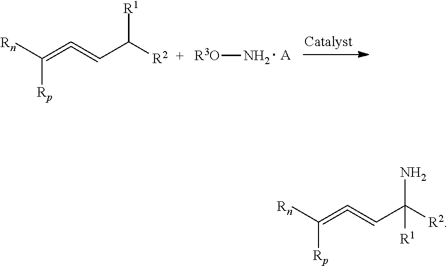

Further, the reaction conditions are: reacting in a mixed solvent of dioxane and water at 20-60° C. for 12-48 h.

Further, said R is substituted or unsubstituted straight or branched alkyl, and R¹ and R² are each independently selected from any group;

the substituent is selected from any one or more of halogen, ester group, alkoxy, phenyl, and benzyl.

Further, R is straight or branched C₁₋₁₈ alkyl, and R¹ and R² are each independently selected from the group consisting of H or straight or branched C₁₋₁₈ alkyl;

preferably, R is straight or branched C₁₋₁₀ alkyl, and R¹ and R² are each independently selected from the group consisting of H or straight C₁₋₁₀ alkyl.

Further, R and R¹ are linked to form a ring, and R² is H.

Further, R and R¹ are linked to form a saturated ring, and the reaction scheme is as follows:

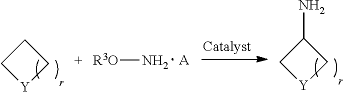

wherein, Y represents CHR″, r represents any integer from 0 to 12; R″ represents any group, and is preferably selected from the group consisting of H, methyl, ethyl, or benzyl.

Further, the reaction conditions are: reacting in a mixed solvent of dioxane and water at 25-80° C. for 12-48 h.

Further, the structure of said compound 1 is selected from the group consisting of following structures:

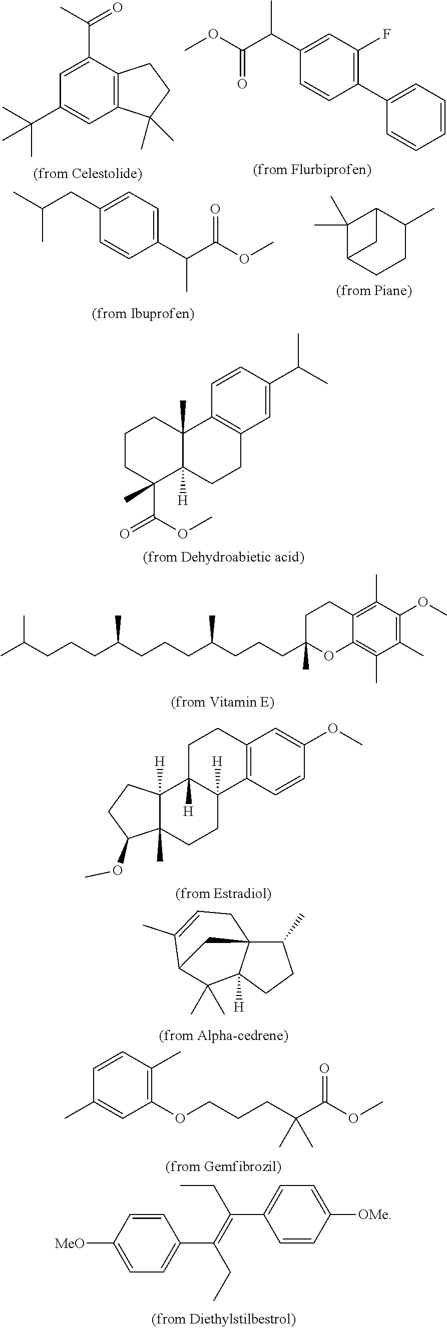

Further, R, R¹, and R² are linked to form a bridged ring.

Further, compound 1 is selected from

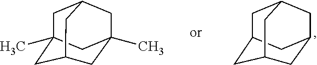

and the reaction scheme is as follows:

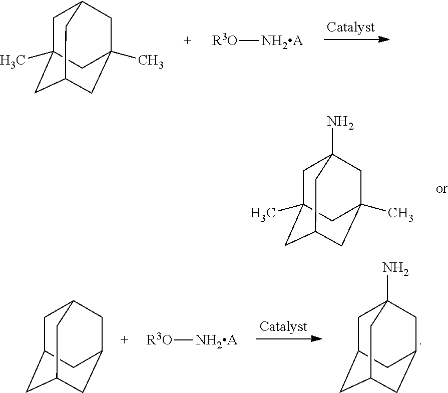

Further, the reaction conditions are: reacting in a mixed solvent of dioxane and water at 20-80° C. for 12-72 h, and preferably reacting at 25° C. for 12 h.

Further, R represents substituted or unsubstituted alkynyl.

Further, said R is

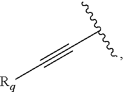

wherein R_{q} is any group.

Further, R¹ and R² are each independently any group, and the reaction scheme is as follows:

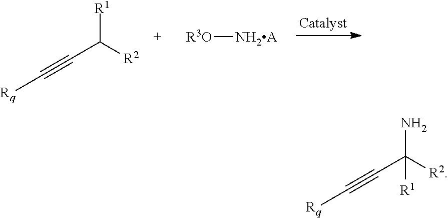

Further, R¹ and R² are independently selected from the group consisting of H, phenyl, and straight or branched C₁₋₁₀ alkyl; R_{q} is selected from the group consisting of H, phenyl, and straight or branched C₁₋₁₀ alkyl.

Further, the reaction conditions are: reacting in a mixed solvent of dioxane and water at 20-60° C. for 12-48 h.

Further, the structure of said compound 1 is selected from the group consisting of:

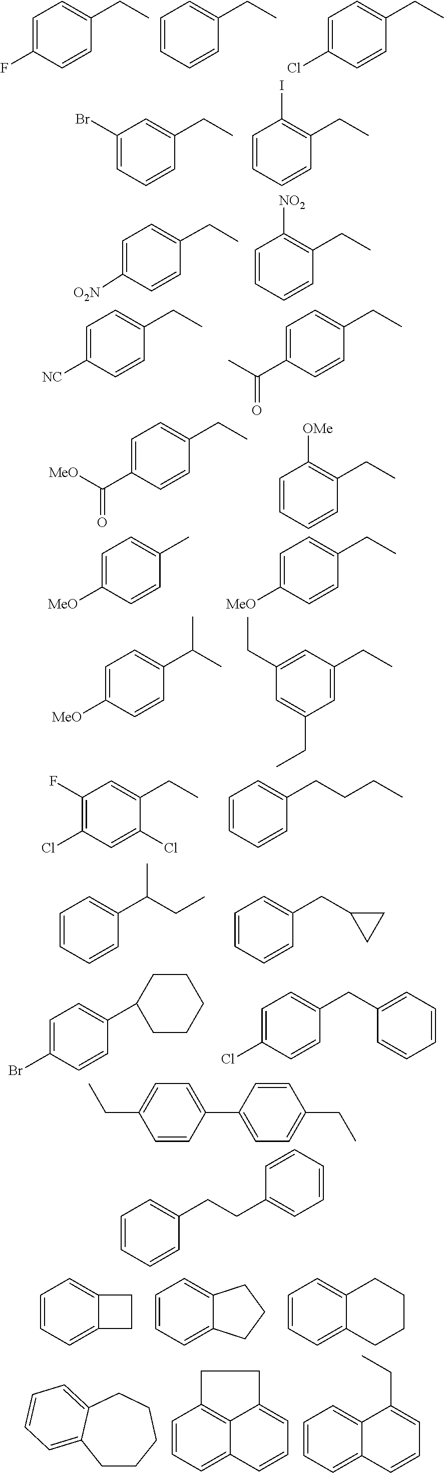

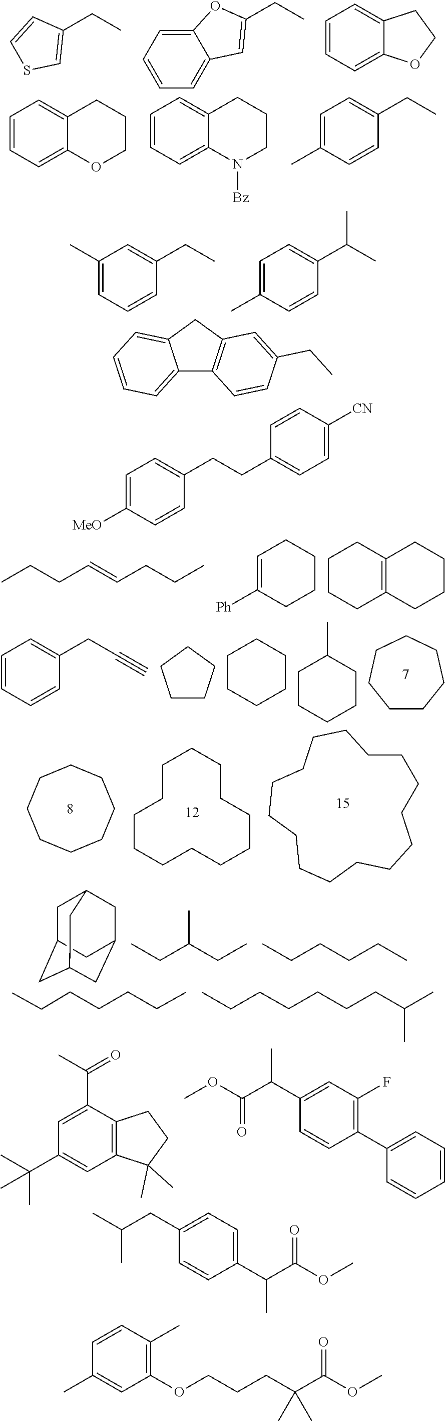

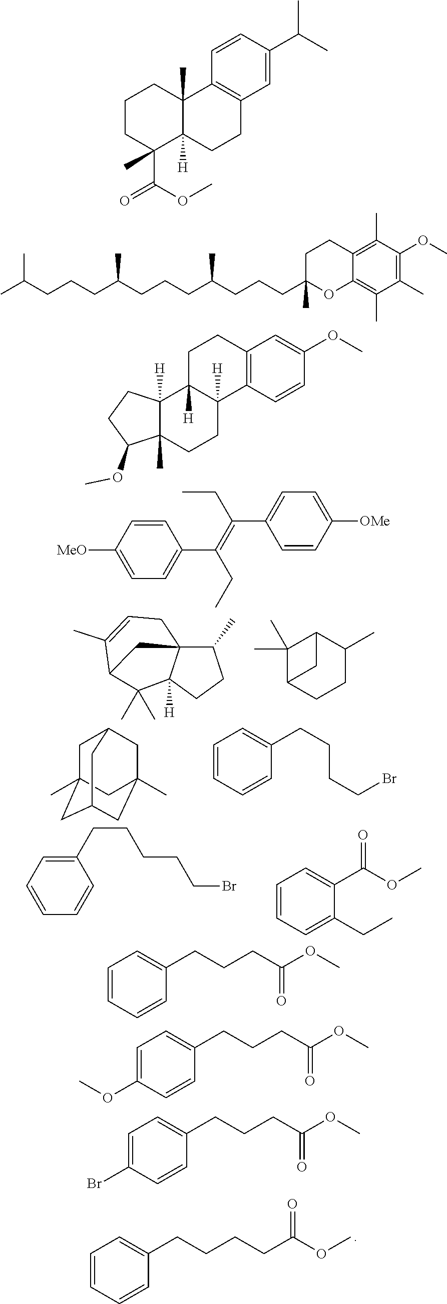

The present invention also provides a method for synthesizing cyclic secondary amines or lactams, which comprises the step of cyclizing the primary amine compound prepared by the method described above under the action of a base, to produce a cyclic secondary amine or lactam compound. In the above method for preparing primary amine compounds, R represents substituted or unsubstituted straight or branched alkyl, and R¹ and R² are each independently selected from any group; the substituent is selected from the group consisting of halogen, ester group, alkoxy, phenyl, and benzyl.

Further, R represents substituted C₁₋₅ straight alkyl, and R¹ and R² are each independently selected from any group; the substituent is selected from the group consisting of halogen or ester group;
the reaction scheme is as follows:

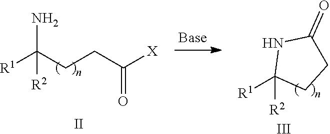

wherein, X is selected from the group consisting of halogen or alkoxy, and n is an integer ranging from 1 to 3.

Further, R¹ is substituted or unsubstituted phenyl, R² is hydrogen, and n is 1 or 2; the substituent is selected from the group consisting of halogen or methyl ester group.

For the term of the present invention: “Any two or three of R, R¹, and R² are linked to form a substituted or unsubstituted ring”, “ring” includes saturated or unsaturated carbocycle, heterocycle, fused ring, bridged ring, etc.

“Sulfonic acid” refers to acids containing sulfonic acid groups, including methanesulfonic acid, ethanesulfonic acid, trifluoromethanesulfonic acid, etc.

“Piv” represents pivaloyl.

Beneficial effects of the present invention:
1. The present invention provides an environmental-friendly chemical method for synthesizing primary amine compounds from sp³-carbon-hydrogen bonds using a specific catalyst. The reaction exhibits broad substrate applicability, not only for conventional sp³-carbon-hydrogen bond compounds at the benzyl, allyl, and propargyl positions, but also for sp³-carbon-hydrogen bonds in alkanes. The primary amine product can be obtained by separation and purification;
2. The method of the present invention can be applied to the direct primary amination modification of complex natural products or active pharmaceutical molecules, as well as the direct synthesis of some important pharmaceutical molecules (such as memantine), and it can be used to achieve further intramolecular tandem cyclization reactions, enabling the efficient synthesis of cyclic secondary amines and lactam compounds;
3. The reaction in the present invention does not need to be carried out under anaerobic conditions. Most reactions can be performed under mild conditions of room temperature and exposure to air. The reaction is easy to operate and has potential industrial application prospects.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. The structures of catalysts C1-C22 selected in Experimental Example 1.

FIG. 2. A standard curve plotted by quantitative analysis to calculate the analytical yield of the product in Experimental Example 1.

### EXAMPLES

The raw materials and equipment used in the present invention are all known products, obtained by purchasing commercially available products.

In the present invention, the catalyst iron octachlorophthalocyanine can be synthesized by known methods. There are two specific ways to use it: one is to use it directly; the other is to immobilize iron octachlorophthalocyanine on silica gel to obtain an immobilized catalyst, which can then be used directly.

Synthesis method for supported catalyst iron octachlorophthalocyanine: 21.1 mg (5 mol %) of catalyst iron octachlorophthalocyanine was weighed and transferred into a round-bottom flask, to which was added 70 mL of dry dichloromethane, and then subjected to ultrasonic treatment for 5 min, followed by addition of 2 g of silica gel (70-200 meshes). The reaction mixture was stirred and reacted at 25° C. for 3 h. The solvent was removed by rotatory evaporation under reduced pressure. The resultant black-green solid was dried in vacuum at 85° C. for 12 h to obtain supported catalyst iron octachlorophthalocyanine.

In the example of the present invention, the separation yield=the amount of target product purified and separated by column chromatography/the theoretical production amount of target product×100%.

In the example of the present invention, unless otherwise specified, the purity of the separated product was greater than 99%.

In the example of the present invention, “Catalyst X mol %” indicated that the amount of catalyst accounted for the molar percentage (X %) based on compound 1.

### General Synthetic Process I

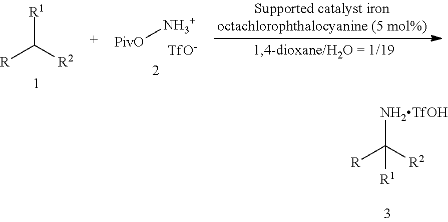

To a dry round-bottom flask, were added the supported catalyst iron octachlorophthalocyanine (5 mol %, supported on 2 g of silica gel), compound 1 (0.5 mmol), dioxane (2 mL), and the solution of N-source 2 (1.5 mmol) in water (38 mL) in sequence. The reaction solution was stirred and reacted at 20-100° C. for 12-120 h. The reaction was monitored by TLC or GC-MS until compound 1 was completely consumed, to provide the primary amine product. Then, the solvent was rotatory evaporated to dry under reduced pressure. The residue was purified by column chromatography to obtain primary amine product 3 (in the form of trifluoromethanesulfonate).

### General Synthetic Process II

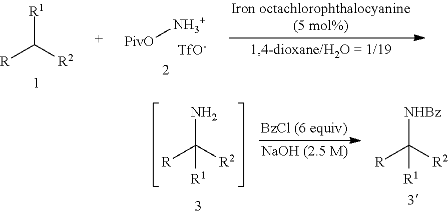

To a dry round-bottom flask, were added the catalyst iron octachlorophthalocyanine (5 mol %), compound 1 (0.5 mmol), dioxane (2 mL), and the solution of N-source 2 (1.5 mmol) in water (38 mL) in sequence. The reaction solution was stirred and reacted at 20-100° C. for 12-120 h. The reaction was monitored by TLC or GC-MS until compound 1 was completely consumed, to provide primary amine product 3. In an ice-bath, aqueous NaOH solution and benzoyl chloride were added in sequence. The reaction was stirred at room temperature, and monitored via TLC or GC-MS until the conversion of the primary amination product in the first step was complete. The aqueous phase was extracted with ethyl acetate. The organic phase was successively washed with saturated NaHCO₃ aqueous solution and saturated saline, and then rotatory evaporated to dry under reduced pressure. The residue was purified by column chromatography to obtain the benzoyl-protected product 3′ (Note: In some cases, protecting the primary amine product with benzoyl could yield higher separation yields).

Representative specific examples were as follows:

### Example 1. Synthesis of

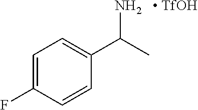

Using general synthetic process I, the reaction solution was stirred and reacted at 25° C. for 12 h, and after workup, 109.9 mg of primary amine salt was obtained. Appearance: white solid; isolation yield: 76%. Melting point: 96-98° C.; ¹H NMR (400 MHz, CD₃OD): δ (ppm) 8.34-8.10 (m, 2H), 8.01-7.82 (m, 2H), 5.61 (s, 3H, NH₃), 5.21 (q, J=6.9 Hz, 1H), 2.35 (d, J=6.9 Hz, 3H); ¹³C NMR (100 MHz, CD₃OD): δ (ppm) 163.7 (d, J=246.9 Hz), 134.9 (d, J=3.3 Hz), 129.3 (d, J=8.5 Hz), 121.1 (q, J=318.2 Hz), 116.4 (d, J=22.0 Hz), 51.0, 19.8; ¹⁹F NMR (376 MHz, CD₃OD): δ (ppm) −79.40, −113.89-−113.98 (m); HRMS (ESI-TOF) m/z: [M-TfO]⁺ Calcd for C₈H₁₁FN⁺ 140.0870; Found 140.0867.

### Example 2. Synthesis of

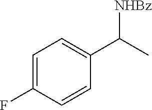

Using general synthetic process II, the reaction solution was stirred and reacted at 25° C. for 12 h, and after workup, 103.4 mg of benzoyl-protected product was obtained. Appearance: white solid; isolation yield: 85%. Melting point: 110-112° C.; ¹H NMR (400 MHz, CDCl₃): δ (ppm) 7.76 (d, J=7.0 Hz, 2H), 7.54-7.47 (m, 1H), 7.46-7.40 (m, 2H), 7.40-7.34 (m, 2H), 7.13-6.89 (m, 2H), 6.28 (d, J=7.6 Hz, 1H), 5.46-5.22 (m, 1H), 1.60 (d, J=6.9 Hz, 3H); ¹³C NMR (100 MHz, CDCl₃): δ (ppm) 166.6, 162.0 (d, ¹J_{FC}=245.5 Hz), 139.0 (d, ⁴J_{FC}=3.4 Hz), 134.4, 131.6, 128.6, 127.9 (d, ³J_{FC}=8.1 Hz), 126.9, 115.5 (d, ²J_{FC}=21.4 Hz), 48.6, 21.8; ¹⁹F NMR (376 MHz, CDCl₃): δ (ppm) −115.13-−115.31 (m); HRMS (ESI-TOF) m/z: Calcd for C₁₅H₁₄FNO+Na⁺ 266.0952, found 266.0948.

### Example 3. Synthesis of

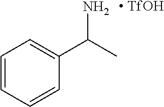

Using general synthetic process I, the reaction solution was stirred and reacted at 25° C. for 12 h, and after workup, 109.6 mg of primary amine salt was obtained. Appearance: white solid; isolation yield: 81%. Melting point: 115-117° C.; ¹H NMR (400 MHz, CD₃OD): δ (ppm) 7.52-7.33 (m, 5H), 4.92 (s, 3H, NH₃), 4.46 (q, J=6.9 Hz, 1H), 1.63 (d, J=6.9 Hz, 3H); ¹³C NMR (100 MHz, CD₃OD): δ (ppm) 138.1, 128.9, 128.8, 126.3, 120.4 (q, J=318.4 Hz), 51.0, 19.3; ¹⁹F NMR (376 MHz, CD₃OD): δ (ppm) −80.03; HRMS (ESI-TOF) m/z: [M-TfO]⁺ Calcd for C₈H₁₂N⁺ 122.0964; Found 122.0958.

### Example 4. Synthesis of

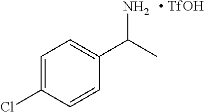

Using general synthetic process I, the reaction solution was stirred and reacted at 25° C. for 12 h, and after workup, 109.8 mg of primary amine salt was obtained. Appearance: white solid; isolation yield: 72%. Melting point: 141-143° C.; ¹H NMR (400 MHz, CD₃OD): δ (ppm) 7.53-7.38 (m, 4H), 4.89 (s, 3H, NH₃), 4.48 (q, J=6.8 Hz, 1H), 1.62 (d, J=6.8 Hz, 3H); ¹³C NMR (100 MHz, CD₃OD): δ (ppm) 136.8, 134.7, 129.0, 128.1, 120.4 (q, J=318.3 Hz), 50.3, 19.1; ¹⁹F NMR (376 MHz, CD₃OD): δ (ppm) −80.03; HRMS (ESI-TOF) m/z: [M-TfO]⁺ Calcd for C₈H₁₁³⁵ClN⁺ 156.0575; Found 156.0582; Calcd for C₈H₁₁³⁷ClN⁺ 158.0546; Found 158.0550.

### Example 5. Synthesis of

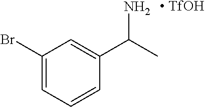

Using general synthetic process I, the reaction solution was stirred and reacted at 25° C. for 12 h, and after workup, 120.9 mg of primary amine salt was obtained. Appearance: white solid; isolation yield: 69%. Melting point: 104-106° C.; ¹H NMR (400 MHz, CD₃OD): δ (ppm) 7.62 (s, 1H), 7.50 (d, J=8.0 Hz, 1H), 7.40 (d, J=8.0 Hz, 1H), 7.32 (t, J=7.8 Hz, 1H), 4.96 (s, 3H, NH₃), 4.29 (q, J=6.8 Hz, 1H), 1.52 (d, J=6.8 Hz, 3H); ¹³C NMR (100 MHz, CD₃OD): δ (ppm) 143.9, 131.0, 130.5, 129.2, 125.0, 122.4, 120.4 (q, J=318.5 Hz), 50.4, 20.9; ¹⁹F NMR (376 MHz, CD₃OD): δ (ppm) −79.98; HRMS (ESI-TOF) m/z: [M-TfO]⁺ Calcd for C₈H₁₁⁷⁹BrN⁺ 200.0070; Found 200.0072; Calcd for C₈H₁₁⁸¹BrN⁺ 202.0049; Found 202.0048.

### Example 6. Synthesis of

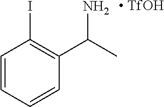

Using general synthetic process I, the reaction solution was stirred and reacted at 25° C. for 12 h, and after workup, 135.6 mg of primary amine salt was obtained. Appearance: white solid; isolation yield: 68%. Melting point: 170-172° C.; ¹H NMR (400 MHz, CD₃OD): δ (ppm) 7.82 (d, J=7.9 Hz, 1H), 7.50 (d, J=7.9 Hz, 1H), 7.39 (t, J=7.6 Hz, 1H), 6.96 (t, J=7.6 Hz, 1H), 4.87 (s, 3H, NH₃), 4.30 (q, J=6.6 Hz, 1H), 1.34 (d, J=6.6 Hz, 3H); ¹³C NMR (100 MHz, CD₃OD): δ (ppm) 148.2, 139.4, 128.6, 128.5, 125.7, 98.2, 54.7, 22.6; ¹⁹F NMR (376 MHz, CD₃OD): δ (ppm) −80.11; HRMS (ESI-TOF) m/z: [M-TfOH+Na]⁺ Calcd for C₈H₁₀INNa⁺ 269.9750; Found 269.9742.

### Example 7. Synthesis of

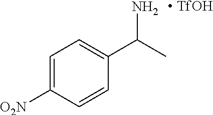

Using general synthetic process I, the reaction solution was stirred and reacted at 40° C. for 12 h, and after workup, 118.9 mg of primary amine salt was obtained. Appearance: white solid; isolation yield: 75%. Melting point: 133-135° C.; ¹H NMR (400 MHz, CD₃OD): δ (ppm) 8.32 (d, J=8.8 Hz, 2H), 7.75 (d, J=8.8 Hz, 2H), 4.87 (s, 3H, NH₃), 4.67 (q, J=6.9 Hz, 1H), 1.70 (d, J=6.9 Hz, 3H); ¹³C NMR (100 MHz, CD₃OD): δ (ppm) 148.2, 145.0, 127.8, 123.9, 120.4 (q, J=317.9 Hz), 50.3, 19.1; ¹⁹F NMR (376 MHz, CD₃OD): δ (ppm) −79.98; HRMS (ESI-TOF) m/z: [M-TfO]⁺ Calcd for C₈H₁₁N₂O₂⁺ 167.0815; Found 167.0820.

### Example 8. Synthesis of

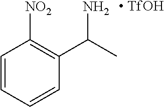

Using general synthetic process I, the reaction solution was stirred and reacted at 60° C. for 12 h, and after workup, the crude product was obtained, and then dissolved in 5 mL of dichloromethane, to which was added trifluoromethanesulfonic acid (48 μL, 0.6 mmol). The reaction was stirred at room temperature for 1 h. The solvent was removed by rotatory evaporation under reduced pressure. The residue was purified by column chromatography, to obtain 108.5 mg of primary amine salt. Appearance: brown liquid; isolation yield: 69%; ¹H NMR (400 MHz, CD₃OD): δ (ppm) 8.06 (d, J=8.2 Hz, 1H), 7.89-7.74 (m, 2H), 7.71-7.59 (m, 1H), 4.98-4.93 (m, 1H), 4.92 (s, 3H, NH₃), 1.69 (d, J=6.8 Hz, 3H); ¹³C NMR (100 MHz, CD₃OD): δ (ppm) 148.7, 134.1, 132.4, 130.1, 127.4, 125.1, 120.4 (q, J=318.3 Hz), 46.0, 18.6; ¹⁹F NMR (376 MHz, CD₃OD): δ (ppm) −80.10; HRMS (ESI-TOF) m/z: [M-TfO]⁺ Calcd for C₈H₁₁N₂O₂⁺ 167.0815; Found 167.0808.

### Example 9. Synthesis of

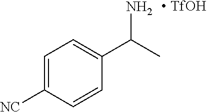

Using general synthetic process I, the reaction solution was stirred and reacted at 40° C. for 12 h, and after workup, 103.3 mg of primary amine salt was obtained. Appearance: colorless liquid; isolation yield: 70%; ¹H NMR (400 MHz, CD₃OD): δ (ppm) 7.69 (d, J=8.4 Hz, 2H), 7.55 (d, J=8.4 Hz, 2H), 4.87 (s, 3H, NH₃), 4.11 (q, J=6.8 Hz, 1H), 1.39 (d, J=6.8 Hz, 3H); ¹³C NMR (100 MHz, CD₃OD): δ (ppm) 152.6, 132.1, 126.7, 118.4, 110.2, 50.7, 23.6; ¹⁹F NMR (376 MHz, CD₃OD): δ (ppm) −80.12; HRMS (ESI-TOF) m/z: [M-TfO]⁺ Calcd for C₉H₁₁N₂⁺ 147.0917; Found 147.0926.

### Example 10. Synthesis of

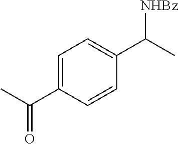

Using general synthetic process II, the reaction solution was stirred and reacted at 60° C. for 24 h. Due to the formation of imine by-products, after workup, the crude product was dissolved in 15 mL mixed solution of dioxane and water (1:1), and then stirred and reacted at 40° C. overnight. After completion of the reaction, NaOH aqueous solution was added to adjust the pH to 10, and TLC monitoring was conducted until the imine by-products were completely converted. The aqueous phase was extracted with ethyl acetate, and then rotatory evaporated to dry under reduced pressure. The residue was purified by column chromatography, to provide 62.8 mg of benzoyl-protected product. Appearance: white solid; separation yield: 47%. Melting point: 169-171° C.; ¹H NMR (400 MHz, CDCl₃): δ (ppm) 7.99-7.86 (m, 2H), 7.85-7.74 (m, 2H), 7.53-7.38 (m, 5H), 6.62 (d, J=7.7 Hz, 1H), 5.43-5.28 (m, 1H), 2.57 (s, 3H), 1.60 (d, J=6.9 Hz, 3H); ¹³C NMR (100 MHz, CDCl₃): δ(ppm) 197.7, 166.8, 148.8, 136.2, 134.2, 131.7, 128.8, 128.6, 127.0, 126.7, 126.3, 49.2, 46.0, 26.6, 21.9; HRMS (ESI-TOF) m/z: [M+Na]⁺ Calcd for C₁₇H₁₇NNaO₂⁺ 290.1151; Found 290.1149.

### Example 11. Synthesis of

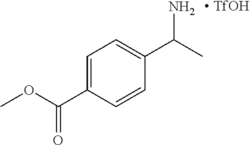

Using general synthetic process I, the reaction solution was stirred and reacted at 25° C. for 16 h, and after workup, 139.3 mg of primary amine salt was obtained. Appearance: white solid; isolation yield: 85%. M.p. 122-124° C.; ¹H NMR (400 MHz, CD₃CN): δ (ppm) 8.08 (d, J=8.4 Hz, 2H), 7.94 (s, 3H, NH₃), 7.59 (d, J=8.4 Hz, 2H), 4.71-4.54 (m, 1H), 3.91 (s, 3H), 1.66 (d, J=6.9 Hz, 3H); ¹³C NMR (100 MHz, CD₃CN): δ (ppm) 167.0, 145.3-140.6 (m), 131.8, 130.7, 128.1, 52.7, 52.6-52.2 (m), 24.4-16.4 (m); 19F NMR (376 MHz, CD₃CN): δ (ppm) −79.51; HRMS (ESI-TOF) m/z: [M−TfO]⁺ Calcd for C₁₀H₁₄NO₂⁺ 180.1019; Found 180.1022.

### Example 12. Synthesis of

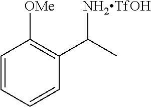

Using general synthetic process I, the reaction solution was stirred and reacted at 25° C. for 12 h, and after workup, 121.6 mg of primary amine salt was obtained. Appearance: white solid; isolation yield: 81%. M.p. 111-113° C.; ¹H NMR (400 MHz, CD₃OD): δ (ppm) 7.43-7.26 (m, 2H), 7.09 (d, J=8.3 Hz, 1H), 7.02 (t, J=7.5 Hz, 1H), 4.98 (s, 3H, NH₃), 4.64 (q, J=6.9 Hz, 1H), 3.91 (s, 3H), 1.62 (d, J=6.9 Hz, 3H); ¹³C NMR (100 MHz, CD₃OD): δ (ppm) 157.0, 130.3, 127.1, 125.3, 120.7, 120.4 (q, J=318.5 Hz), 111.0, 54.7, 47.1, 17.5; ¹⁹F NMR (376 MHz, CD₃OD): δ (ppm) −80.01; HRMS (ESI-TOF) m/z: [M-TfO]⁺ Calcd for C₉H₁₄NO⁺ 152.1070; Found 152.1070.

### Example 13. Synthesis of

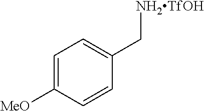

Using general synthetic process I, the reaction solution was stirred and reacted at 25° C. for 12 h, and after workup, 105.6 mg of primary amine salt was obtained. Appearance: white solid; isolation yield: 74%. M.p. 92-94° C.; ¹H NMR (400 MHz, CD₃OD): δ (ppm) 7.38 (d, J=8.7 Hz, 2H), 6.97 (d, J=8.7 Hz, 2H), 4.95 (s, 3H, NH₃), 4.04 (s, 2H), 3.80 (s, 3H); ¹³C NMR (100 MHz, CD₃OD): δ (ppm) 160.5, 130.2, 124.8, 120.4 (q, J=318.4 Hz), 114.1, 54.4, 42.6; ¹⁹F NMR (376 MHz, CD₃OD): δ (ppm) −80.03; HRMS (ESI-TOF) m/z: [M-TfO]⁺ Calcd for C₈H₁₂NO⁺ 138.0913; Found 138.0917.

### Example 14. Synthesis of

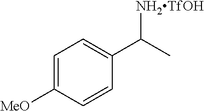

Using general synthetic process I, the reaction solution was stirred and reacted at 25° C. for 12 h, and after workup, 109.3 mg of primary amine salt was obtained. Appearance: white solid; isolation yield: 73%. M.p. 114-116° C.; ¹H NMR (400 MHz, CD₃OD): δ (ppm) 7.39 (d, J=8.7 Hz, 2H), 6.98 (d, J=8.7 Hz, 2H), 4.88 (brs, 3H, NH₃), 4.41 (q, J=6.9 Hz, 1H), 3.80 (s, 3H), 1.61 (d, J=6.9 Hz, 3H); ¹³C NMR (100 MHz, CD₃OD): δ (ppm) 160.3, 129.9, 127.8, 120.4 (q, J=318.4 Hz), 114.2, 54.5, 50.5, 19.1; ¹⁹F NMR (376 MHz, CD₃OD): δ (ppm) −80.01; HRMS (ESI-TOF) m/z: [M-TfO]⁺ Calcd for C₉H₁₄NO⁺ 152.1070; Found 152.1071.

### Example 15. Synthesis of

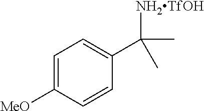

Using general synthetic process I, the reaction solution was stirred and reacted at 25° C. for 24 h, and after workup, 126.2 mg of primary amine salt was obtained. Appearance: yellow liquid; isolation yield: 80%; ¹H NMR (400 MHz, CD₃OD): δ (ppm) 7.45 (d, J=8.9 Hz, 2H), 6.99 (d, J=8.9 Hz, 2H), 4.89 (brs, 3H, NH₃), 3.80 (s, 3H), 1.71 (s, 6H); ¹³C NMR (100 MHz, CD₃OD): δ (ppm) 159.8, 133.3, 125.8, 120.4 (q, J=318.4 Hz), 113.9, 55.3, 54.4, 26.6; ¹⁹F NMR (376 MHz, CD₃OD): δ (ppm) −80.06; HRMS (ESI-TOF) m/z: [M-TfO]⁺ Calcd for C₁₀H₁₆NO⁺ 166.1226; Found 166.1225.

### Example 16. Synthesis of

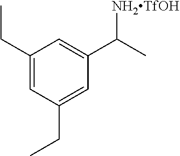

Using general synthetic process I, the reaction solution was stirred and reacted at 25° C. for 12 h, and after workup, 123.4 mg of primary amine salt was obtained. Appearance: semi solid; isolation yield: 75%; ¹H NMR (400 MHz, CD₃OD): δ (ppm) 7.21-6.94 (m, 3H), 4.99 (s, 3H, NH₃), 4.32 (q, J=6.8 Hz, 1H), 2.64 (q, J=7.6 Hz, 4H), 1.57 (d, J=6.8 Hz, 3H), 1.23 (t, J=7.6 Hz, 6H); ¹³C NMR (100 MHz, CD₃OD): δ (ppm) 145.2, 139.8, 127.5, 122.9, 120.4 (q, J=318.4 Hz), 51.1, 28.4, 20.3, 14.8; ¹⁹F NMR (376 MHz, CD₃OD): δ (ppm) −79.97; HRMS (ESI-TOF) m/z: [M-TfO]⁺ Calcd for C₁₂H₂₀N⁺ 178.1590; Found 178.1583.

### Example 17. Synthesis of

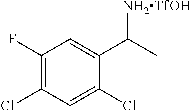

Using general synthetic process I, the reaction solution was stirred and reacted at 60° C. for 12 h, and after workup, the crude product was obtained, and then dissolved in 5 mL of dichloromethane, to which was added trifluoromethanesulfonic acid (48 μL, 0.6 mmol) in an ice bath. The reaction was stirred at room temperature for 1 h. The solvent was removed by rotatory evaporation under reduced pressure. The residue was purified by column chromatography, to obtain 90.2 mg of primary amine salt. Appearance: white solid; isolation yield: 50%. M.p. 110-112° C.; ¹H NMR (400 MHz, CD₃OD): δ (ppm) 7.75 (d, J=6.8 Hz, 1H), 7.50 (d, J=9.8 Hz, 1H), 4.89-4.78 (m, 4H, NH₃+CH), 1.62 (d, J=6.9 Hz, 3H); ¹³C NMR (100 MHz, CD₃OD): δ (ppm) 157.2 (d, J=249.7 Hz), 136.4 (d, J=6.3 Hz), 131.6, 128.3 (d, J=3.8 Hz), 122.1 (d, J=19.0 Hz), 120.4 (q, J=318.5 Hz), 114.9 (d, J=24.3 Hz), 47.1, 18.1; ¹⁹F NMR (376 MHz, CD₃OD): δ (ppm) −80.10, −110.07-−125.97 (m); HRMS (ESI-TOF) m/z: [M-TfOH+Na]⁺ Calcd for C₈H₈³⁵Cl₂FNNa⁺ 229.9911; Found 229.9913; Calcd for C₈H₈³⁷Cl₂FNNa⁺ 231.9881; Found 231.9887.

### Example 18. Synthesis of

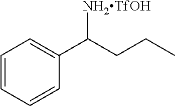

Using general synthetic process I, the reaction solution was stirred and reacted at 25° C. for 12 h, and after workup, 103.3 mg of primary amine salt was obtained. Appearance: white solid; isolation yield: 69%. M.p. 122-124° C.; ¹H NMR (400 MHz, CD₃OD): δ (ppm) 7.54-7.29 (m, 5H), 4.97 (s, 3H, NH₃), 4.23 (dd, J=9.0 Hz, 6.2 Hz, 1H), 2.04-1.82 (m, 2H), 1.35-1.11 (m, 2H), 0.94 (t, J=7.4 Hz, 3H); ¹³C NMR (100 MHz, CD₃OD): δ (ppm) 139.5, 128.7, 128.2, 126.7, 120.4 (q, J=318.4 Hz), 55.5, 37.7, 18.8, 12.6; ¹⁹F NMR (376 MHz, CD₃OD): δ (ppm) −79.96; HRMS (ESI-TOF) m/z: [M-TfO]⁺ Calcd for C₁₀H₁₆N⁺ 150.1277; Found 150.1275.

### Example 19. Synthesis of

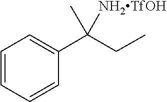

Using general synthetic process I, the reaction solution was stirred and reacted at 25° C. for 12 h, and after workup, 99.3 mg of primary amine salt was obtained. Appearance: white solid; isolation yield: 66%. M.p. 97-99° C.; ¹H NMR (400 MHz, CD₃OD): δ (ppm) 7.56-7.33 (m, 5H), 5.00 (s, 3H, NH₃), 2.13-1.97 (m, 2H), 1.70 (s, 3H), 0.80 (t, J=7.5 Hz, 3H); ¹³C NMR (100 MHz, CD₃OD): δ (ppm) 140.3, 128.7, 128.0, 124.9, 120.4 (q, J=318.4 Hz), 59.0, 33.8, 23.6, 7.0; ¹⁹F NMR (376 MHz, CD₃OD): δ (ppm) −80.02; HRMS (ESI-TOF) m/z: [M-TfO]⁺ Calcd for C₁₀H₁₆N⁺ 150.1277; Found 150.1275.

### Example 20. Synthesis of

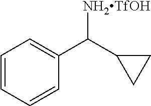

Using general synthetic process I, the reaction solution was stirred and reacted at 25° C. for 12 h, and after workup, 128.4 mg of primary amine salt was obtained. Appearance: colorless liquid; isolation yield: 86%; ¹H NMR (400 MHz, CD₃OD): δ (ppm) 7.55-7.28 (m, 5H), 4.98 (s, 3H, NH₃), 3.46 (d, J=9.7 Hz, 1H), 1.38-1.29 (m, 1H), 0.81-0.70 (m, 1H), 0.65-0.48 (m, 2H), 0.43-0.29 (m, 1H); ¹³C NMR (100 MHz, CD₃OD): δ (ppm) 137.5, 127.3, 126.9, 125.3, 119.1 (q, J=318.5 Hz), 59.0, 14.3, 2.6, 1.4; ¹⁹F NMR (376 MHz, CD₃OD): δ (ppm) −79.98; HRMS (ESI-TOF) m/z: [M-TfO]⁺ Calcd for C₁₀H₁₄N⁺ 148.1121; Found 148.1126.

### Example 21. Synthesis of

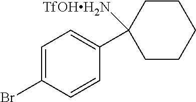

Using general synthetic process I, the reaction solution was stirred and reacted at 25° C. for 12 h, and after workup, 150.3 mg of primary amine salt was obtained. Appearance: colorless liquid; isolation yield: 74%; ¹H NMR (400 MHz, CD₃OD): δ (ppm) 7.52 (d, J=8.8 Hz, 2H), 7.46 (d, J=8.8 Hz, 2H), 4.90 (s, 3H, NH₃), 2.24-2.02 (m, 2H), 1.85-1.63 (m, 4H), 1.59-1.38 (m, 4H); ¹³C NMR (100 MHz, CD₃OD): δ (ppm) 144.3, 131.3, 127.7, 120.7, 120.4 (q, J=318.5 Hz), 54.6, 37.0, 25.1, 21.9; ¹⁹F NMR (376 MHz, CD₃OD): δ (ppm) −79.95; HRMS (ESI-TOF) m/z: [M-TfOH+Na]⁺ Calcd for C₁₂H₁₆⁷⁹BrNNa⁺ 276.0359; Found 276.0361; Calcd for C₁₂H₁₆⁸¹BrNNa⁺ 278.0338; Found 278.0346.

### Example 22. Synthesis of

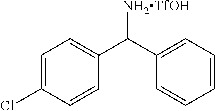

Using general synthetic process I, the reaction solution was stirred and reacted at 25° C. for 24 h, and after workup, the crude product was obtained, and then dissolved in 5 mL of dichloromethane, to which was added trifluoromethanesulfonic acid (48 μL, 0.6 mmol) in an ice bath. The reaction was stirred at room temperature for 1 h. The solvent was removed by rotatory evaporation under reduced pressure. The residue was purified by column chromatography, to obtain 140.1 mg of primary amine. Appearance: brown solid; isolation yield: 76%. M.p. 144-146° C.; ¹H NMR (400 MHz, CD₃OD): δ (ppm) 7.42-7.29 (m, 8H), 7.29-7.23 (m, 1H), 5.27 (s, 1H), 4.90 (s, 3H, NH₃); ¹³C NMR (100 MHz, CD₃OD): δ (ppm) 142.3, 141.6, 132.9, 128.44, 128.41, 128.35, 127.4, 126.7, 120.4 (d, J=318.6 Hz), 58.3; ¹⁹F NMR (376 MHz, CD₃OD): δ (ppm) −80.01; HRMS (ESI-TOF) m/z: [M-TfO]⁺ Calcd for C₁₃H₁₃³⁵ClN⁺ 218.0732; Found 218.0734; Calcd for C₁₃H₁₃³⁷ClN⁺ 220.0702; Found 220.0697.

### Example 23. Synthesis of

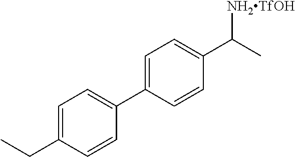

Using general synthetic process I, the reaction solution was stirred and reacted at 80° C. for 24 h, and after workup, 140.7 mg of primary amine salt was obtained. Appearance: yellow solid; isolation yield: 75%. M.p. 184-186° C.; ¹H NMR (400 MHz, CD₃OD): δ (ppm) 7.68 (d, J=8.4 Hz, 2H), 7.61-7.45 (m, 4H), 7.28 (d, J=8.2 Hz, 2H), 4.95 (s, 3H, NH₃), 4.47 (q, J=6.8 Hz, 1H), 2.67 (q, J=7.6 Hz, 2H), 1.65 (d, J=6.9 Hz, 3H), 1.25 (t, J=7.6 Hz, 3H); ¹³C NMR (100 MHz, CD₃OD): δ(ppm) 143.8, 141.8, 137.4, 137.3, 128.1, 127.1, 126.7, 126.5, 120.4 (q, J=318.4 Hz), 50.7, 28.1, 19.5, 14.8; ¹⁹F NMR (376 MHz, CD₃OD): δ (ppm) −80.01; HRMS (ESI-TOF) m/z: [M-TfOH+Na]⁺ Calcd for C₁₆H₁₉NNa⁺ 248.1410; Found 248.1417.

### Example 24. Synthesis of

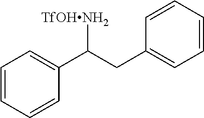

Using general synthetic process I, the reaction solution was stirred and reacted at 25° C. for 16 h, and after workup, the crude product was obtained, and then dissolved in 5 mL of dichloromethane, to which was added trifluoromethanesulfonic acid (48 μL, 0.6 mmol) in an ice bath. The reaction was stirred at room temperature for 1 h. The solvent was removed by rotatory evaporation under reduced pressure. The residue was purified by column chromatography, to obtain 144.9 mg of primary amine. Appearance: colorless liquid; isolation yield: 83%; ¹H NMR (400 MHz, CD₃OD): & (ppm) 7.34-7.25 (m, 4H), 7.25-7.18 (m, 3H), 7.17-7.12 (m, 1H), 7.08 (d, J=6.8 Hz, 2H), 4.87 (s, 3H, NH₃), 4.10 (t, J=7.2 Hz, 1H), 2.96 (d, J=7.2 Hz, 2H); ¹³C NMR (100 MHz, CD₃OD): δ (ppm) 144.1, 138.5, 129.1, 128.01, 127.95, 126.8, 126.4, 126.0, 57.5, 45.3; ¹⁹F NMR (376 MHz, CD₃OD): δ (ppm) −80.06; HRMS (ESI-TOF) m/z: [M-TfO]⁺ Calcd for C₁₄H₁₆N⁺ 198.1277; Found 198.1279.

### Example 25. Synthesis of

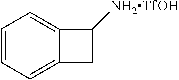

Using general synthetic process I, the reaction solution was stirred and reacted at 25° C. for 24 h, and after workup, 90.1 mg of primary amine salt was obtained. Appearance: white solid; isolation yield: 67%. M.p. 136-138° C.; ¹H NMR (400 MHz, CD₃OD): δ (ppm) 7.39 (t, J=7.2 Hz, 1H), 7.36-7.25 (m, 2H), 7.21 (d, J=7.2 Hz, 1H), 4.91 (s, 3H, NH₃), 4.78 (dd, J=5.0 Hz, 2.1 Hz, 1H), 3.68 (dd, J=14.6 Hz, 5.0 Hz, 1H), 3.22 (dd, J=14.6 Hz, 2.1 Hz, 1H); ¹³C NMR (100 MHz, CD₃OD): δ (ppm) 142.2, 140.7, 130.4, 127.8, 123.3, 122.6, 49.5, 36.0; ¹⁹F NMR (376 MHz, CD₃OD): δ (ppm) −80.09; HRMS (ESI-TOF) m/z: [M-TfO]⁺ Calcd for C₈H₁₀N⁺ 120.0808; Found 120.0801.

### Example 26. Synthesis of

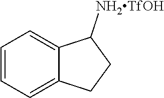

Using general synthetic process I, the reaction solution was stirred and reacted at 25° C. for 12 h, and after workup, 112.7 mg of primary amine salt was obtained. Appearance: white solid; isolation yield: 80%. M.p. 143-145° C.; ¹H NMR (400 MHz, CD₃OD): δ (ppm) 7.48 (d, J=7.4 Hz, 1H), 7.42-7.32 (m, 2H), 7.33-7.24 (m, 1H), 4.89 (s, 3H, NH₃), 4.76 (dd, J=7.8 Hz, 5.0 Hz, 1H), 3.21-3.11 (m, 1H), 3.09-2.93 (m, 1H), 2.68-2.53 (m, 1H), 2.15-1.98 (m, 1H); ¹³C NMR (100 MHz, CD₃OD): δ (ppm) 144.0, 138.6, 129.3, 126.9, 125.0, 124.0, 55.6, 30.4, 29.6; ¹⁹F NMR (376 MHz, CD₃OD): δ (ppm) −79.98; HRMS (ESI-TOF) m/z: [M-TfO]⁺ Calcd for C₉H₁₂N⁺ 134.0964; Found 134.0966.

### Example 27. Synthesis of

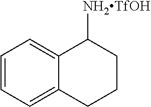

Using general synthetic process I, the reaction solution was stirred and reacted at 25° C. for 12 h, and after workup, 121.9 mg of primary amine salt was obtained. Appearance: white solid; isolation yield: 82%. M.p. 116-118° C.; ¹H NMR (400 MHz, CD₃OD): δ (ppm) 7.47-7.35 (m, 1H), 7.34-7.10 (m, 3H), 4.99 (s, 3H, NH₃), 4.47 (t, J=5.4 Hz, 1H), 3.06-2.62 (m, 2H), 2.28-2.06 (m, 1H), 2.06-1.72 (m, 3H); ¹³C NMR (100 MHz, CD₃OD): δ (ppm) 137.7, 131.8, 129.5, 128.5, 128.1, 126.3, 120.4 (q, J=318.5 Hz), 48.9, 28.3, 27.8, 18.1; ¹⁹F NMR (376 MHz, CD₃OD): δ (ppm) −80.00; HRMS (ESI-TOF) m/z: [M-TfO]⁺ Calcd for C₁₀H₁₄N⁺ 148.1121; Found 148.1129.

### Example 28. Synthesis of

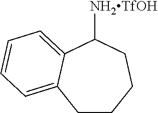

Using general synthetic process I, the reaction solution was stirred and reacted at 25° C. for 12 h, and after workup, 116.9 mg of primary amine salt was obtained. Appearance: white solid; isolation yield: 75%. M.p. 162-164° C.; ¹H NMR (400 MHz, CD₃OD): δ (ppm) 7.30-7.12 (m, 4H), 4.95 (s, 3H, NH₃), 4.47 (dd, J=10.0 Hz, 1.6 Hz, 1H), 2.94-2.78 (m, 2H), 2.08-1.81 (m, 4H), 1.74-1.58 (m, 1H), 1.45-1.33 (m, 1H); ¹³C NMR (100 MHz, CD₃OD): δ (ppm) 141.1, 139.1, 129.7, 127.5, 126.2, 122.9, 120.4 (q, J=318.4 Hz), 53.9, 34.9, 33.8, 28.2, 26.8; ¹⁹F NMR (376 MHz, CD₃OD): δ (ppm) −80.03; HRMS (ESI-TOF) m/z: [M-TfO]⁺ Calcd for C₁₁H₁₆N⁺ 162.1277; Found 162.1280.

### Example 29. Synthesis of

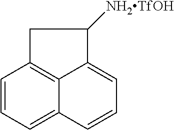

Using general synthetic process I, the reaction solution was stirred and reacted at 60° C. for 12 h, and after workup, 71.8 mg of primary amine salt was obtained. Appearance: semi solid; isolation yield: 45%; ¹H NMR (400 MHz, CD₃OD): δ (ppm) 7.75-7.64 (m, 1H), 7.61 (d, J=8.2 Hz, 1H), 7.56-7.34 (m, 3H), 7.28 (d, J=6.8 Hz, 1H), 5.00-4.77 (m, 4H), 3.75 (dd, J=17.6 Hz, 7.8 Hz, 1H), 3.10 (dd, J=17.6 Hz, 2.9 Hz, 1H); ¹³C NMR (100 MHz, CD₃OD): δ (ppm) 145.6, 141.7, 137.4, 131.5, 127.9, 127.7, 124.0, 122.3, 119.5, 119.3, 54.2, 39.8; ¹⁹F NMR (376 MHz, CD₃OD): δ (ppm) −80.00; HRMS (ESI-TOF) m/z: [M-TfO]⁺ Calcd for C₁₂H₁₂N⁺ 170.0964; Found 170.0964.

### Example 30. Synthesis of

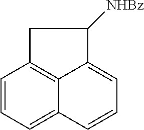

Using general synthetic process II, the reaction solution was stirred and reacted at 60° C. for 12 h. After workup, 101.1 mg of benzoyl-protected product was obtained. Appearance: white solid, isolation yield: 74%. M.p.: 195-197° C.; ¹H NMR (400 MHz, CDCl₃): δ (ppm) 7.76-7.65 (m, 3H), 7.61 (d, J=8.2 Hz, 1H), 7.48-7.41 (m, 3H), 7.39-7.32 (m, 2H), 7.27 (d, J=6.8 Hz, 1H), 7.19 (s, 1H), 6.48 (d, J=8.1 Hz, 1H), 6.17-5.99 (m, 1H), 3.98 (dd, J=17.8 Hz, 7.9 Hz, 1H), 3.18 (dd, J=17.8 Hz, 3.2 Hz, 1H); ¹³C NMR (100 MHz, CDCl₃): δ (ppm) 167.1, 144.2, 142.1, 137.9, 134.3, 131.7, 131.3, 128.6, 128.4, 128.1, 127.0, 124.8, 122.9, 120.2, 120.0, 53.3, 40.5; HRMS (ESI-TOF) m/z: [M+Na]⁺ Calcd for C₁₉H₁₅NNaO⁺ 296.1046; Found 296.1051.

### Example 31. Synthesis of

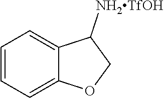

Using general synthetic process I, the reaction solution was stirred and reacted at 25° C. for 12 h, and after workup, 117.8 mg of primary amine salt was obtained. Appearance: brown solid; isolation yield: 83%. M.p. 172-174° C.; ¹H NMR (400 MHz, CD₃OD): δ (ppm) 7.52 (d, J=7.5 Hz, 1H), 7.36 (t, J=8.1 Hz, 1H), 7.02 (t, J=7.5 Hz, 1H), 6.93 (d, J=8.1 Hz, 1H), 5.03 (dd, J=7.6 Hz, 2.7 Hz, 1H), 4.92 (s, 3H, NH₃), 4.69 (dd, J=11.3 Hz, 7.6 Hz, 1H), 4.55 (dd, J=11.3 Hz, 2.7 Hz, 1H); ¹³C NMR (100 MHz, CD₃OD): δ (ppm) 160.7, 131.5, 125.5, 122.4, 121.2, 120.4 (d, J=318.3 Hz), 110.4, 73.8, 52.4; ¹⁹F NMR (376 MHz, CD₃OD): δ (ppm) −80.09; HRMS (ESI-TOF) m/z: [M-TfO]⁺ Calcd for C₈H₁₀NO⁺ 136.0757; Found 136.0758.

### Example 32. Synthesis of

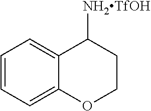

Using general synthetic process I, the reaction solution was stirred and reacted at 25° C. for 16 h, and after workup, 106.2 mg of primary amine salt was obtained. Appearance: white solid; isolation yield: 71%. M.p. 169-171° C.; ¹H NMR (400 MHz, CD₃OD): δ (ppm) 7.35 (d, J=7.8 Hz, 1H), 7.22 (t, J=7.6 Hz, 1H), 6.95 (t, J=7.6 Hz, 1H), 6.83 (d, J=7.8 Hz, 1H), 4.95 (s, 3H, NH₃), 4.39 (t, J=5.3 Hz, 1H), 4.25 (dd, J=6.8 Hz, 4.2 Hz, 2H), 2.40-2.21 (m, 1H), 2.16-2.01 (m, 1H); ¹³C NMR (100 MHz, CD₃OD): δ (ppm) 154.9, 129.7, 128.7, 120.6, 120.4 (q, J=318.6 Hz), 119.9, 117.1, 61.6, 44.6, 27.8; ¹⁹F NMR (376 MHz, CD₃OD): δ (ppm) −80.04; HRMS (ESI-TOF) m/z: [M-TfO]⁺ Calcd for C₉H₁₂NO⁺ 150.0913; Found 150.0908.

### Example 33. Synthesis of

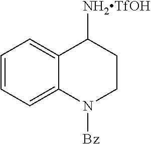

Using general synthetic process I, the reaction solution was stirred and reacted at 60° C. for 24 h, and after workup, the crude product was obtained, and then dissolved in 5 mL of dichloromethane, to which was added trifluoromethanesulfonic acid (48 μL, 0.6 mmol) in an ice bath. The reaction was stirred at room temperature for 1 h. The solvent was removed by rotatory evaporation under reduced pressure. The residue was purified by column chromatography, to obtain 142.3 mg of primary amine. Appearance: colorless liquid; isolation yield: 71%; ¹H NMR (400 MHz, CD₃OD): δ (ppm) 7.54-7.27 (m, 6H), 7.16 (t, J=7.6 Hz, 1H), 7.01 (t, J=7.8 Hz, 1H), 6.86 (d, J=8.2 Hz, 1H), 4.90 (s, 3H, NH₃), 4.47-4.32 (m, 1H), 4.10-3.98 (m, 1H), 3.98-3.79 (m, 1H), 2.51-2.31 (m, 1H), 2.00-1.86 (m, 1H); ¹³C NMR (100 MHz, CD₃OD): δ (ppm) 171.1, 138.2, 135.8, 130.4, 130.1, 128.13, 128.10, 127.3, 126.6, 125.3, 125.1, 120.4 (q, J=318.7 Hz), 47.0, 42.1, 30.6; ¹⁹F NMR (376 MHz, CD₃OD): δ (ppm) −80.02; HRMS (ESI-TOF) m/z: [M-TfO]⁺ Calcd for C₁₆H₁₇N₂O⁺ 253.1335; Found 253.1344.

### Example 34. Synthesis of

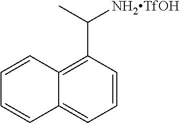

Using general synthetic process I, the reaction solution was stirred and reacted at 25° C. for 12 h, and after workup, 85.9 mg of primary amine salt was obtained. Appearance: semi solid; isolation yield: 53%; ¹H NMR (400 MHz, CD₃OD): δ (ppm) 8.12 (d, J=8.5 Hz, 1H), 7.92 (d, J=8.5 Hz, 1H), 7.87 (d, J=8.2 Hz, 1H), 7.73-7.46 (m, 4H), 5.20 (q, J=6.7 Hz, 1H), 4.92 (s, 3H, NH₃), 1.66 (d, J=6.7 Hz, 3H); ¹³C NMR (100 MHz, CD₃OD): δ (ppm) 137.3, 134.1, 130.2, 128.8, 128.4, 126.5, 125.7, 125.1, 122.0, 121.8, 120.4 (d, J=318.4 Hz), 46.0, 21.1; ¹⁹F NMR (376 MHz, CD₃OD): δ (ppm) −80.04; HRMS (ESI-TOF) m/z: [M-TfO]⁺ Calcd for C₁₂H₁₄N⁺ 172.1121; Found 172.1123.

### Example 35. Synthesis of

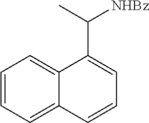

Using general synthetic process II, the reaction solution was stirred and reacted at 25° C. for 12 h. After workup, 87.8 mg of benzoyl-protected product was obtained. Appearance: white solid, isolation yield: 64%. M.p.: 164-166° C.; ¹H NMR (400 MHz, CDCl₃): δ (ppm) 8.14 (d, J=8.1 Hz, 1H), 7.87-7.82 (m, 1H), 7.79 (d, J=8.2 Hz, 1H), 7.71 (d, J=7.6 Hz, 2H), 7.57 (d, J=7.2 Hz, 1H), 7.53-7.39 (m, 4H), 7.33 (t, J=7.6 Hz, 2H), 6.51 (d, J=8.0 Hz, 1H), 6.16-6.03 (m, 1H), 1.74 (d, J=6.8 Hz, 3H); ¹³C NMR (100 MHz, CDCl₃): δ (ppm) 166.5, 138.3, 134.5, 134.0, 131.5, 131.3, 128.8, 128.54, 128.51, 127.0, 126.7, 125.9, 125.3, 123.5, 122.7, 45.3, 20.7; HRMS (ESI-TOF) m/z: [M+Na]⁺ Calcd for C₁₉H₁₇NNaO⁺ 298.1202; Found 298.1208.

### Example 36. Synthesis of

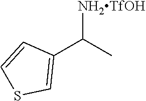

Using general synthetic process I, the reaction solution was stirred and reacted at 25° C. for 24 h, and after workup, 101.8 mg of primary amine salt was obtained. Appearance: brown liquid; isolation yield: 73%; ¹H NMR (400 MHz, CD₃OD): δ (ppm) 7.47 (dd, J=5.1 Hz, 1.2 Hz, 1H), 7.21 (d, J=3.6 Hz, 1H), 7.07 (dd, J=5.1 Hz, 3.6 Hz, 1H), 4.89 (s, 3H, NH₃), 4.74 (q, J=6.8 Hz, 1H), 1.68 (d, J=6.8 Hz, 3H); ¹³C NMR (100 MHz, CD₃OD): δ (ppm) 140.3, 127.0, 126.5, 126.2, 46.2, 19.7; 19F NMR (376 MHz, CD₃OD): δ (ppm) −80.03; HRMS (ESI-TOF) m/z: [M-TfOH+Na]⁺ Calcd for C₆H₉NNaS⁺ 150.0348; Found 150.0340.

### Example 37. Synthesis of

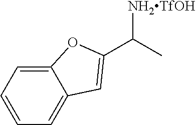

Using general synthetic process I, the reaction solution was stirred and reacted at 25° C. for 12 h, and after workup, 84.2 mg of primary amine salt was obtained. Appearance: semi solid; isolation yield: 54%; ¹H NMR (400 MHz, CD₃OD): δ (ppm) 7.57 (d, J=7.6 Hz, 1H), 7.48 (d, J=8.2 Hz, 1H), 7.29 (t, J=8.2 Hz, 1H), 7.22 (t, J=7.6 Hz, 1H), 6.79 (s, 1H), 4.96 (s, 3H, NH₃), 4.47 (q, J=6.8 Hz, 1H), 1.63 (d, J=6.8 Hz, 3H); ¹³C NMR (100 MHz, CD₃OD): δ (ppm) 157.0, 155.0, 128.0, 124.3, 122.8, 121.0, 120.4 (q, J=318.5 Hz), 110.6, 103.0, 44.8, 18.0; ¹⁹F NMR (376 MHz, CD₃OD): δ (ppm) −79.99; HRMS (ESI-TOF) m/z: [M-TfOH+Na]⁺ Calcd for C₁₀H₁₁NNaO⁺ 184.0733; Found 184.0724.

### Example 38. Synthesis of

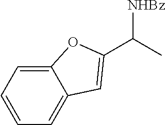

Using general synthetic process II, the reaction solution was stirred and reacted at 25° C. for 12 h. After workup, 82.0 mg of benzoyl-protected product was obtained. Appearance: white solid, isolation yield: 62%. M.p.: 131-133° C.; ¹H NMR (400 MHz, CDCl₃): δ (ppm) 7.82-7.76 (m, 2H), 7.54-7.35 (m, 5H), 7.28-7.15 (m, 2H), 6.69 (d, J=8.4 Hz, 1H), 6.59 (s, 1H), 5.62-5.45 (m, 1H), 1.65 (d, J=6.9 Hz, 3H); ¹³C NMR (100 MHz, CDCl₃): δ (ppm) 166.7, 158.0, 154.8, 134.2, 131.7, 128.6, 128.2, 127.1, 124.2, 122.9, 121.0, 111.2, 102.7, 43.8, 19.7; HRMS (ESI-TOF) m/z: [M+Na]⁺ Calcd for C₁₇H₁₅NNaO₂⁺ 288.0995; Found 288.0998.

### Example 39. Synthesis of

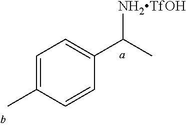

Using general synthetic process I, the reaction solution was stirred and reacted at 25° C. for 12 h, and after workup, 121.7 mg of primary amine salt was obtained. Appearance: semi solid; isolation yield: 85%, 11:1 r.r. (a:b); ¹H NMR (400 MHz, CD₃OD): δ (ppm) 7.33 (d, J=8.0 Hz, 2H), 7.26 (d, J=8.0 Hz, 2H), 4.97 (s, 3H, NH₃), 4.41 (q, J=6.9 Hz, 1H), 2.35 (s, 3H), 1.60 (d, J=6.9 Hz, 3H); ¹³C NMR (100 MHz, CD₃OD): δ (ppm) 138.9, 135.2, 129.5, 126.2, 120.4 (q, J=318.4 Hz), 50.8, 19.8, 19.3; ¹⁹F NMR (376 MHz, CD₃OD) δ (ppm) −80.10; HRMS (ESI-TOF) m/z: [M-TfO]⁺ Calcd for C₉H₁₄N⁺ 136.1121; Found 136.1126.

### Example 40. Synthesis of

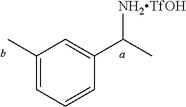

Using general synthetic process I, the reaction solution was stirred and reacted at 25° C. for 12 h, and after workup, 110.9 mg of primary amine salt was obtained. Appearance: semi solid; isolation yield: 78%; 11:1 r.r. (a:b); ¹H NMR (400 MHz, CD₃CN): δ (ppm) 7.40-7.21 (m, 4H), 5.91 (s, 3H, NH₃), 4.49 (q, J=6.8 Hz, 1H), 2.39 (s, 3H), 1.63 (d, J=6.8 Hz, 3H); ¹³C NMR (100 MHz, CD₃CN): δ (ppm) 139.5, 138.1, 130.4, 129.5, 128.1, 124.4, 121.3 (q, J=319.7 Hz), 52.7, 21.0, 20.0; ¹⁹F NMR (376 MHz, CD₃CN) δ (ppm) −79.35; HRMS (ESI-TOF) m/z: [M-TfO]⁺ Calcd for C₉H₁₄N⁺ 136.1121; Found 136.1124.

### Example 41. Synthesis of

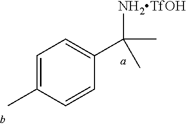

Using general synthetic process I, the reaction solution was stirred and reacted at 25° C. for 12 h, and after workup, 98.8 mg of primary amine salt was obtained. Appearance: semi solid; isolation yield: 66%, >19:1 r.r. (a:b); ¹H NMR (400 MHz, CD₃OD): δ (ppm) 7.28 (d, J=8.4 Hz, 2H), 7.16 (d, J=8.4 Hz, 2H), 4.82 (s, 3H, NH₃), 2.24 (s, 3H), 1.59 (s, 6H); ¹³C NMR (100 MHz, CD₃OD): δ (ppm) 139.3, 138.0, 129.2, 124.2, 120.4 (q, J=318.6 Hz), 55.2, 27.1, 19.6; ¹⁹F NMR (376 MHz, CD₃OD): δ (ppm) −80.09; HRMS (ESI-TOF) m/z: [M-TfO]⁺ Calcd for C₁₀H₁₆N⁺ 150.1277; Found 150.1275.

### Example 42. Synthesis of

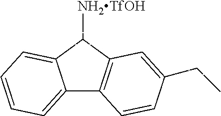

Using general synthetic process I, the reaction solution was stirred and reacted at 25° C. for 16 h, and after workup, 152.3 mg of primary amine salt was obtained. Appearance: white solid; isolation yield: 85%. M.p. 232-234° C.; 7:1 r.r. (a:b); ¹H NMR (400 MHz, CD₃OD): δ (ppm) 7.74-7.56 (m, 3H), 7.54-7.48 (m, 1H), 7.33 (t, J=7.6 Hz, 1H), 7.26 (t, J=7.4 Hz, 1H), 7.19 (d, J=7.8 Hz, 1H), 4.88 (s, 3H, NH₃), 4.76 (s, 1H), 2.70 (q, J=7.6 Hz, 2H), 1.27 (t, J=7.6 Hz, 3H); ¹³C NMR (100 MHz, CD₃OD): δ (ppm) 147.4, 147.0, 143.9, 140.2, 137.7, 127.8, 127.5, 126.7, 124.2, 123.8, 119.3, 119.1, 56.9, 28.7, 15.0; ¹⁹F NMR (376 MHz, CD₃OD) δ (ppm) −80.01; HRMS (ESI-TOF) m/z: [M-TfO]⁺ Calcd for C₁₅H₁₆N⁺ 210.1277; Found 210.1273.

### Example 43. Synthesis of

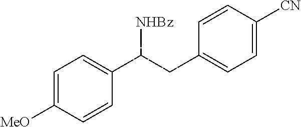

Using general synthetic process II, the reaction solution was stirred and reacted at 60° C. for 120 h. After workup, 78.4 mg of benzoyl-protected product was obtained. Appearance: white solid, isolation yield: 44% (Total separation yield of regioisomers). M.p.: 181-183° C.; 3:1 r.r. (a:b); regioisomer mixture: ¹H NMR (400 MHz, CDCl₃): δ (ppm) 7.73-7.67 (m, 2.6H), 7.50-7.54 (m, 0.6H), 7.52-7.45 (m, 3.3H), 7.44-7.33 (m, 3.3H), 7.24-7.12 (m, 4.0H), 6.96 (d, J=8.3 Hz, 0.6H), 6.8-6.77 (m, 2.6H), 5.41-5.29 (m, 1.3H), 3.83-3.70 (m, 4.0H), 3.38 (dd, J=13.4, 6.5 Hz, 1.0H), 3.24-3.06 (m, 1.6H); ¹³C NMR (100 MHz, CDCl₃): δ (ppm) 167.0, 166.9, 159.2, 158.7, 147.4, 143.4, 134.3, 133.9, 132.4, 132.1, 131.9, 131.7, 130.24, 130.17, 128.69, 128.65, 128.0, 127.4, 127.0, 126.9, 118.9, 118.8, 114.24, 114.16, 111.1, 110.4, 55.31, 55.25, 54.8, 54.6, 42.4, 41.5; HRMS (ESI-TOF) m/z: [M+Na]⁺ Calcd for C₂₃H₂₀N₂NaO₂⁺ 379.1417; Found 379.1425.

### Example 44. Synthesis of

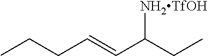

Using general synthetic process I, the reaction solution was stirred and reacted at 25° C. for 24 h, and after workup, 36.0 mg of primary amine salt was obtained. Appearance: yellow liquid; isolation yield: 26%; ¹H NMR (400 MHz, CD₃CN): δ (ppm) 5.92-5.79 (m, 1H), 5.42-5.31 (m, 1H), 4.54 (brs, 3H, NH₃), 3.67-3.55 (m, 1H), 2.13-1.99 (m, 2H), 1.79-1.68 (m, 1H), 1.68-1.52 (m, 1H), 1.48-1.35 (m, 2H), 0.96-0.82 (m, 6H); ¹³C NMR (100 MHz, CD₃CN): δ (ppm) 139.0, 125.4, 121.5 (q, J=320.0 Hz), 56.8, 34.5, 26.4, 22.2, 13.4, 9.7; ¹⁹F NMR (376 MHz, CD₃CN): δ (ppm) −78.60; HRMS (ESI-TOF) m/z: [M-TfOH+Na]⁺ Calcd for C₈H₁₇NNa⁺ 150.1253; Found 150.1261.

### Example 45. Synthesis of

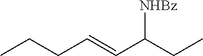

Using general synthetic process II, the reaction solution was stirred and reacted at 25° C. for 24 h. After workup, 36.0 mg of benzoyl-protected product was obtained. Appearance: semi solid, isolation yield: 31%; ¹H NMR (400 MHz, CDCl₃): δ (ppm) 7.84-7.70 (m, 2H), 7.61-7.36 (m, 3H), 5.98 (d, J=8.5 Hz, 1H), 5.78-5.59 (m, 1H), 5.50-5.32 (m, 1H), 4.56 (q, J=7.1 Hz, 1H), 2.02 (q, J=7.1 Hz, 2H), 1.70-1.63 (m, 2H), 1.44-1.35 (m, 2H), 0.96 (t, J=7.4 Hz, 3H), 0.89 (t, J=7.4 Hz, 3H); ¹³C NMR (100 MHz, CDCl₃): δ (ppm) 166.7, 135.0, 132.1, 131.3, 129.9, 128.6, 126.9, 52.7, 34.4, 28.4, 22.3, 13.7, 10.3; HRMS (ESI-TOF) m/z: [M+Na]⁺ Calcd for C₁₅H₂₁NNaO⁺ 254.1515; Found 254.1524.

### Example 46. Synthesis of

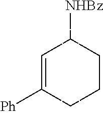

Using general synthetic process II, the reaction solution was stirred and reacted at 40° C. for 24 h. After workup, 62.0 mg of benzoyl-protected product was obtained. Appearance: white solid, isolation yield: 45%. M.p.: 135-137° C.; 9:1 r.r. (a:b); ¹H NMR (400 MHz, CDCl₃): δ (ppm) 7.79-7.67 (m, 2H), 7.46-7.39 (m, 1H), 7.34 (dd, J=8.5, 6.9 Hz, 4H), 7.25 (dd, J=8.5, 6.5 Hz, 2H), 7.22-7.17 (m, 1H), 6.15 (d, J=8.2 Hz, 1H), 5.99 (s, 1H), 4.92-4.77 (m, 1H), 2.49-2.30 (m, 2H), 2.06-1.97 (m, 1H), 1.83-1.74 (m, 2H), 1.6-1.58 (m, 1H); ¹³C NMR (100 MHz, CDCl₃): δ (ppm) 166.8, 141.2, 140.7, 134.8, 131.4, 128.6, 128.4, 127.5, 126.9, 125.3, 124.4, 46.0, 29.2, 27.3, 20.4; HRMS (ESI-TOF) m/z: [M+Na]⁺ Calcd for C₁₉H₁₉NNaO⁺ 300.1359; Found 300.1350. C₁₉H₁₉NNaO⁺ 300.1359; Found 300.1350.

### Example 47. Synthesis of

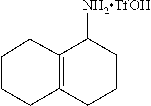

Using general synthetic process I, the reaction solution was stirred and reacted at 25° C. for 12 h, and after workup, 97.4 mg of primary amine salt was obtained. Appearance: white solid; isolation yield: 65%. M.p. 158-160° C.; ¹H NMR (400 MHz, CD₃OD): δ (ppm) 4.88 (s, 3H, NH₃), 3.58 (t, J=5.4 Hz, 1H), 2.18-1.83 (m, 7H), 1.82-1.51 (m, 7H); ¹³C NMR (100 MHz, CD₃OD): δ (ppm) 136.9, 123.5, 120.4 (q, J=318.4 Hz), 50.4, 30.1, 29.6, 27.7, 26.5, 22.4, 22.1, 17.9; ¹⁹F NMR (376 MHz, CD₃OD): δ (ppm) −80.10; HRMS (ESI-TOF) m/z: [M-TfO]⁺ Calcd for C₁₀H₁₈N⁺ 152.1434; Found 152.1430.

### Example 48. Synthesis of

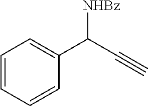

Using general synthetic process II, the reaction solution was stirred and reacted at 25° C. for 12 h. After workup, 12.9 mg of benzoyl-protected product was obtained. Appearance: semi solid, isolation yield: 11%; ¹H NMR (400 MHz, CD₃CN): δ (ppm) 7.76-7.68 (m, 2H), 7.58-7.51 (m, 3H), 7.48-7.37 (m, 5H), 6.53 (d, J=8.3 Hz, 1H), 6.18 (dd, J=8.4, 2.5 Hz, 1H), 2.47 (d, J=2.5 Hz, 1H). (The yield of this compound was relatively low, and it was only characterized by ¹H NMR, which was consistent with the data reported in literature)

### Example 49. Synthesis of

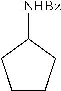

Using general synthetic process II, the reaction solution was stirred and reacted at 25° C. for 12 h. After workup, 25.2 mg of benzoyl-protected product was obtained. Appearance: white solid, isolation yield: 27%. M.p.: 137-139° C.; ¹H NMR (400 MHz, CDCl₃): δ (ppm) 7.79-7.70 (m, 2H), 7.50-7.43 (m, 1H), 7.43-7.35 (m, 2H), 6.24 (brs, 1H), 4.39 (q, J=7.0 Hz, 1H), 2.17-1.96 (m, 2H), 1.78-1.58 (m, 4H), 1.54-1.36 (m, 2H); ¹³C NMR (100 MHz, CDCl₃): δ (ppm) 167.2, 134.9, 131.2, 128.5, 126.9, 51.7, 33.2, 23.8; HRMS (ESI-TOF) m/z: [M+Na]⁺ Calcd for C₁₂H₁₅NNaO⁺ 212.1046; Found 212.1044.

### Example 50. Synthesis of

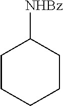

Using general synthetic process II, the reaction solution was stirred and reacted at 25° C. for 12 h. After workup, 9.1 mg of benzoyl-protected product was obtained. Appearance: semi solid, isolation yield: 9%; ¹H NMR (400 MHz, CDCl₃): δ (ppm) 7.74 (d, 2H), 7.5-7.36 (m, 3H), 5.97 (s, 1H), 4.07-3.89 (m, 1H), 2.11-1.95 (m, 2H), 1.81-1.71 (m, 2H), 1.71-1.59 (m, 2H), 1.52-1.33 (m, 2H), 1.31-1.21 (m, 2H); ¹³C NMR (100 MHz, CDCl₃): δ (ppm) 166.6, 135.1, 131.2, 128.5, 126.8, 48.7, 33.3, 25.6, 24.9; HRMS (ESI-TOF) m/z: [M+Na]⁺ Calcd for C₁₃H₁₇NNaO⁺ 226.1202; Found 226.1208.

### Example 51. Synthesis of

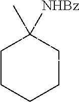

Using general synthetic process II, the reaction solution was stirred and reacted at 25° C. for 16 h. After workup, 62.0 mg of benzoyl-protected product was obtained. Appearance: white solid, isolation yield: 45%. M.p.: 135-137° C.; >10:1 r.r. (a:b); ¹H NMR (400 MHz, CDCl₃): δ (ppm) 7.80-7.64 (m, 2H), 7.54-7.35 (m, 3H), 5.83 (brs, 1H), 2.25-2.00 (m, 2H), 1.65-1.54 (m, 6H), 1.50-1.44 (m, 5H); ¹³C NMR (100 MHz, CDCl₃): δ (ppm) 166.9, 136.2, 131.1, 128.5, 126.7, 53.7, 36.8, 26.5, 25.6, 22.2; HRMS (ESI-TOF) m/z: [M+Na]⁺ Calcd for C₁₄H₁₉NNaO⁺ 240.1359; Found 240.1368.

### Example 52. Synthesis of

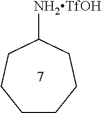

Using general synthetic process I, the reaction solution was stirred and reacted at 25° C. for 12 h, and after workup, 100.1 mg of primary amine salt was obtained. Appearance: white solid; isolation yield: 76%. M.p. 206-208° C.; ¹H NMR (400 MHz, CD₃CN): δ (ppm) 6.38 (s, 3H, NH₃), 3.39-3.27 (m, 1H), 2.04-1.95 (m, 2H), 1.78-1.66 (m, 2H), 1.66-1.35 (m, 8H); ¹³C NMR (100 MHz, CD₃CN): δ (ppm) 54.3, 32.7, 27.8, 23.8; ¹⁹F NMR (376 MHz, CD₃CN) δ (ppm) −79.41; HRMS (ESI-TOF) m/z: [M-TfO]⁺ Calcd for C₇H₁₆N⁺ 114.1277; Found 114.1284.

### Example 53. Synthesis of

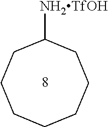

Using general synthetic process I, the reaction solution was stirred and reacted at 25° C. for 12 h, and after workup, 98.2 mg of primary amine salt was obtained. Appearance: white solid; isolation yield: 71%. M.p. 201-203° C.; ¹H NMR (400 MHz, CD₃OD): δ (ppm) 4.90 (s, 3H, NH₃), 3.36-3.31 (m, 1H), 2.03-1.85 (m, 2H), 1.85-1.41 (m, 12H); ¹³C NMR (100 MHz, CD₃OD): δ (ppm) 120.4 (d, J=318.4 Hz), 51.7, 30.5, 26.1, 25.2, 23.1; ¹⁹F NMR (376 MHz, CD₃OD) δ (ppm) −80.10; HRMS (ESI-TOF) m/z: [M-TfO]⁺ Calcd for C₈H₁₈N⁺ 128.1434; Found 128.1433.

### Example 54. Synthesis of

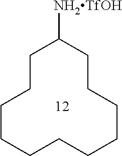

Using general synthetic process I, the reaction solution was stirred and reacted at 40° C. for 24 h, and after workup, 95.0 mg of primary amine salt was obtained. Appearance: white solid; isolation yield: 57%. M.p. 197-199° C.; ¹H NMR (400 MHz, CD₃OD): δ (ppm) 4.88 (s, 3H, NH₃), 3.30-3.24 (m, 1H), 1.88-1.69 (m, 2H), 1.64-1.22 (m, 20H); ¹³C NMR (100 MHz, CD₃OD): δ (ppm) 120.4 (d, J=318.4 Hz), 49.0, 27.7, 23.7, 23.5, 22.8, 22.7, 20.2; ¹⁹F NMR (376 MHz, CD₃OD) δ (ppm) −80.11; HRMS (ESI-TOF) m/z: [M-TfO]⁺ Calcd for C₁₂H₂₆N⁺ 184.2060; Found 184.2065.

### Example 55. Synthesis of

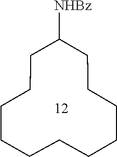

Using general synthetic process II, the reaction solution was stirred and reacted at 40° C. for 24 h. After workup, 113.4 mg of benzoyl-protected product was obtained. Appearance: white solid, isolation yield: 79%. M.p.: 182-184° C.; ¹H NMR (400 MHz, CDCl₃): δ (ppm) 7.75 (d, J=7.4 Hz, 2H), 7.55-7.35 (m, 3H), 5.89 (brs, 1H), 4.30 (d, J=10.1 Hz, 1H), 1.86-1.58 (m, 3H), 1.59-1.10 (m, 19H); ¹³C NMR (100 MHz, CDCl₃): δ (ppm) 166.7, 135.1, 131.3, 128.5, 126.8, 46.6, 30.4, 24.0, 23.8, 23.5, 23.4, 21.5; HRMS (ESI-TOF) m/z: [M+Na]⁺ Calcd for C₁₉H₂₉NNaO⁺ 310.2141; Found 310.2150.

### Example 56. Synthesis of

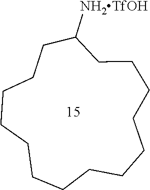

Using general synthetic process I, the reaction solution was stirred and reacted at 60° C. for 16 h, and after workup, 110.8 mg of primary amine salt was obtained. Appearance: white solid; isolation yield: 59%. M.p. 208-210° C.; ¹H NMR (400 MHz, CD₃OD): δ (ppm) 4.95 (s, 3H, NH₃), 3.23-3.16 (m, 1H), 1.75-1.54 (m, 6H), 1.50-1.36 (m, 22H); ¹³C NMR (100 MHz, CD₃OD): δ (ppm) 120.4 (d, J=318.3 Hz), 50.6, 30.7, 26.6, 26.45, 26.40, 26.3, 26.2, 22.7; ¹⁹F NMR (376 MHz, CD₃OD): δ (ppm) −80.09; HRMS (ESI-TOF) m/z: [M-TfO]⁺ Calcd for C₁₅H₃₂N⁺ 226.2529; Found 226.2537.

### Example 57. Synthesis of 4

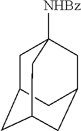

Using general synthetic process II, the reaction solution was stirred and reacted at 80° C. for 48 h. After workup, 12.8 mg of benzoyl-protected product was obtained. Appearance: white solid, isolation yield: 10%, ¹H NMR (400 MHz, CDCl₃): δ (ppm) 7.77-7.66 (m, 2H), 7.50-7.37 (m, 3H), 5.80 (s, 1H), 2.13 (s, 9H), 1.77-1.67 (m, 6H). (The yield of this compound was relatively low, and it was only characterized by ¹H NMR, which was consistent with the data reported in literature)

### Example 58. Synthesis of

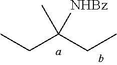

Using general synthetic process II, to a dry round-bottom flask, were sequentially added catalyst iron octachlorophthalocyanine (5 mol %), compound 1 (10.0 mmol), acetonitrile (4 mL) and the aqueous solution of nitrogen source 2 (0.5 mmol) (36 mL). The reaction solution was stirred and reacted at 40° C. for 16 h, and after workup, 21.6 mg of benzoyl-protected product was obtained. Appearance: white solid, isolation yield: 21%. Melting point: 67-69° C.; 5:1 r.r. (a:b); ¹H NMR (400 MHz, CDCl₃): δ (ppm) 7.77-7.65 (m, 2H), 7.52-7.37 (m, 3H), 5.71 (s, 1H), 2.02-1.87 (m, 2H), 1.79-1.72 (m, 2H), 1.34 (s, 3H), 0.89 (t, J=7.5 Hz, 6H); ¹³C NMR (100 MHz, CDCl₃): δ (ppm) 166.9, 136.1, 131.1, 128.5, 126.7, 57.2, 30.5, 23.5, 8.1; HRMS (ESI-TOF) m/z: [M+Na]⁺ Calcd for C₁₃H₁₉NNaO⁺ 228.1359; Found 228.1365.

### Example 59. Synthesis of

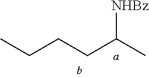

Using general synthetic process II, to a dry round-bottom flask, were sequentially added catalyst iron octachlorophthalocyanine (5 mol %), compound 1 (10.0 mmol), acetonitrile (4 mL) and the aqueous solution of nitrogen source 2 (0.5 mmol) (36 mL). The reaction solution was stirred and reacted at 40° C. for 16 h, and after workup, 12.3 mg of benzoyl-protected product was obtained. Appearance: white solid, isolation yield: 21% (Total separation yield of regioisomers). Melting point: 135-137° C.; 2:1 r.r. (a:b); Regioisomer mixture: ¹H NMR (400 MHz, CDCl₃): δ (ppm) 7.74-7.61 (m, 3H), 7.49-7.30 (m, 4.5H), 5.82 (d, J=8.3 Hz, 1H), 5.74 (d, J=8.9 Hz, 0.5H), 4.19-4.09 (m, 1H), 4.08-3.97 (m, 0.5H), 1.53-1.25 (m, 9H), 1.17 (d, J=6.6 Hz, 3H), 0.93-0.67 (m, 6H); ¹³C NMR (100 MHz, CDCl₃): δ (ppm) 167.3, 166.8, 135.2, 135.1, 131.3, 128.56, 128.54, 126.8, 50.9, 45.8, 37.1, 36.8, 28.3, 28.1, 22.6, 21.1, 19.2, 14.08, 14.03, 10.3; HRMS (ESI-TOF) m/z: [M+Na]⁺ Calcd for C₁₃H₁₉NNaO⁺ 228.1359; Found 228.1363.

### Example 60. Synthesis of

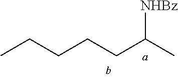

Using general synthetic process II, to a dry round-bottom flask, were sequentially added catalyst iron octachlorophthalocyanine (5 mol %), compound 1 (10.0 mmol), acetonitrile (4 mL) and the aqueous solution of nitrogen source 2 (0.5 mmol) (36 mL). The reaction solution was stirred and reacted at 40° C. for 16 h, and after workup, 14.3 mg of benzoyl-protected regioisomer mixture product was obtained. Appearance: white solid, isolation yield: 13% (Total separation yield of regioisomers). Melting point: 82-84° C.; 2:1 r.r. (a:b); Although regioisomer mixtures could not be completely separated, a portion of pure products could be obtained by purification over column chromatography; isomer a: ¹H NMR (400 MHz, CDCl₃): δ (ppm) 7.75 (d, J=7.5 Hz, 2H), 7.49 (t, J=7.2 Hz, 1H), 7.43 (t, J=7.4 Hz, 2H), 5.87 (d, J=8.2 Hz, 1H), 4.26-4.12 (m, 1H), 1.57-1.50 (m, 2H), 1.41-1.29 (m, 6H), 1.25-1.18 (m, 3H), 0.93-0.86 (m, 3H); ¹³C NMR (100 MHz, CDCl₃): δ (ppm) 166.8, 135.1, 131.3, 128.6, 126.8, 45.8, 37.1, 31.7, 25.8, 22.6, 21.1, 14.0; HRMS (ESI-TOF) m/z: [M+Na]⁺ Calcd for C₁₄H₂₁NNaO⁺ 242.1515; Found 242.1514; isomer b: ¹H NMR (400 MHz, CDCl₃): δ (ppm) 7.78-7.72 (m, 2H), 7.52-7.41 (m, 3H), 5.80 (d, J=9.0 Hz, 1H), 4.14-3.98 (m, 1H), 1.68-1.49 (m, 4H), 1.40-1.32 (m, 4H), 1.00-0.88 (m, 6H); ¹³C NMR (100 MHz, CDCl₃): δ (ppm) 167.2, 135.2, 131.3, 128.6, 126.8, 51.1, 34.6, 28.15, 28.10, 22.7, 14.0, 10.3. HRMS (ESI-TOF) m/z: [M+Na]⁺ Calcd for C₁₄H₂₁NNaO⁺ 242.1515; Found 242.1513.

### Example 61. Synthesis of

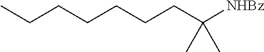

Using general synthetic process II, the reaction solution was stirred and reacted at 25° C. for 12 h. After workup, 23.5 mg of benzoyl-protected product was obtained. Appearance: white solid, isolation yield: 18%. M.p.: 70-72° C.; ¹H NMR (400 MHz, CDCl₃): δ (ppm) 7.77-7.64 (m, 2H), 7.52-7.36 (m, 3H), 5.83 (s, 1H), 1.84-1.74 (m, 2H), 1.59-1.54 (m, 2H), 1.43 (s, 6H), 1.33-1.25 (m, 8H), 0.91-0.82 (m, 3H); ¹³C NMR (100 MHz, CDCl₃): δ (ppm) 166.8, 136.1, 131.1, 128.5, 126.7, 54.2, 40.5, 31.9, 30.0, 29.3, 27.0, 24.2, 22.7, 14.1; HRMS (ESI-TOF) m/z: [M+Na]⁺ Calcd for C₁₇H₂₇NNaO⁺ 284.1985; Found 284.1988.

### General Synthetic Process 3

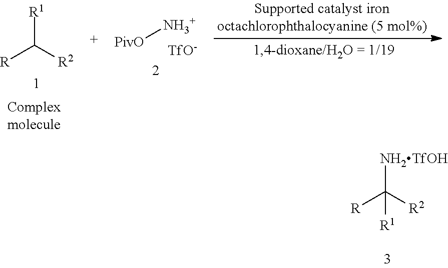

To a dry round-bottom flask, were added the supported catalyst iron octachlorophthalocyanine (5 mol %, supported on 1 g of silica gel), compound 1 (0.25 mmol), dioxane (1 mL), and the aqueous solution of nitrogen source 2 (0.75 mmol) (19 mL) in sequence. The reaction solution was stirred at 20-100° C. for 12-24 h, and the reaction was monitored by TLC or GC-MS until compound 1 was completely consumed, resulting in the formation of primary amine product 3. Then, the solvent was removed by rotatory evaporation under reduced pressure. The residue was purified by column chromatography to obtain the primary amine product (present in the form of trifluoromethanesulfonate).

### Example 62. Synthesis of

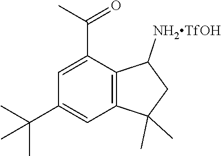

Using general synthetic process 3, the reaction solution was stirred and reacted at 80° C. for 12 h, and after workup, 82.1 mg of primary amine salt was obtained. Appearance: semi-solid, isolation yield: 80%; ¹H NMR (400 MHz, CDCl₃): δ (ppm) 7.87 (d, J=1.8 Hz, 1H), 7.46 (d, J=1.7 Hz, 1H), 6.50 (brs, 3H), 4.97 (dd, J=8.7 Hz, 5.1 Hz, 1H), 2.68 (s, 3H), 2.55 (dd, J=14.5 Hz, 8.7 Hz, 1H), 2.24 (dd, J=14.5 Hz, 5.1 Hz, 1H), 1.44 (s, 3H), 1.37 (s, 9H), 1.25 (s, 3H); ¹³C NMR (100 MHz, CDCl₃): δ (ppm) 203.7, 155.4, 154.5, 133.72, 133.69, 127.8, 125.4, 119.9 (q, J=319.2 Hz), 55.0, 45.2, 43.2, 35.1, 31.2, 30.5, 30.0, 28.0; ¹⁹F NMR (376 MHz, CDCl₃) δ (ppm) −78.62; HRMS (ESI-TOF) m/z: [M-TfO]⁺ Calcd for C₁₇H₂₆NO⁺ 260.2009; Found 260.2003.

### Example 63. Synthesis of

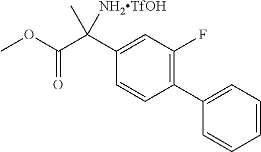

Using general synthetic process 3, the reaction solution was stirred and reacted at 60° C. for 24 h, and after workup, the crude product was obtained, and then dissolved in 5 mL of dichloromethane, to which was added trifluoromethanesulfonic acid (160 μL, 2.0 mmol) in an ice bath. The reaction was stirred at room temperature for 1 h. The solvent was removed by rotatory evaporation under reduced pressure. The residue was purified by column chromatography, to obtain 77.3 mg of primary amine. Appearance: white solid; isolation yield: 73%. M.p.: 152-154° C.; ¹H NMR (400 MHz, CD₃OD): δ (ppm) 7.64 (t, J=8.3 Hz, 1H), 7.58-7.52 (m, 2H), 7.49-7.38 (m, 5H), 4.91 (s, 3H, NH₃), 3.87 (s, 3H), 2.03 (s, 3H); ¹³C NMR (100 MHz, CD₃OD): δ (ppm) 170.3, 159.7 (d, J=248.9 Hz), 136.8 (d, J=7.6 Hz), 134.4, 131.6 (d, J=4.0 Hz), 130.5 (d, J=13.6 Hz), 128.6 (d, J=3.0 Hz), 128.3, 128.1, 121.8 (d, J=3.7 Hz), 120.4 (d, J=318.4 Hz), 113.7 (d, J=25.9 Hz), 61.0, 53.2, 20.8; ¹⁹F NMR (376 MHz, CD₃OD): δ (ppm) −80.06, −117.10-−117.19 (m); HRMS (ESI-TOF) m/z: [M-TfO]⁺ Calcd for C₁₆H₁₇FNO₂⁺ 274.1238; Found 274.1245.

### Example 64. Synthesis of

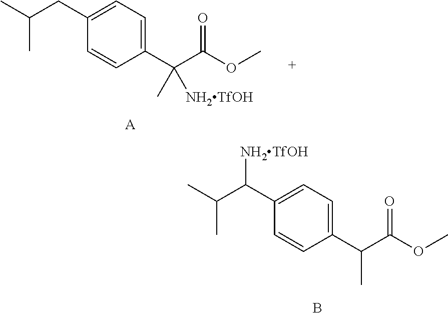

Using general synthetic process 3, the reaction solution was stirred and reacted at 25° C. for 12 h, and after workup, 73.1 mg of primary amine salt was obtained as the regioisomer mixture. Appearance: semi-solid, separation yield: 74% (total separation yield of regioisomers); 1:1.5 r.r. (A/B). Although the regioisomer mixture could not be completely separated, a portion of pure primary amine product could be obtained by purification over column chromatography. Isomer A: ¹H NMR (400 MHz, CDCl₃): δ (ppm) 7.33 (d, J=8.2 Hz, 2H), 7.16 (d, J=8.2 Hz, 2H), 5.92 (brs, 3H), 3.74 (s, 3H), 2.45 (d, J=7.2 Hz, 2H), 1.95 (s, 3H), 1.88-1.79 (m, 1H), 0.88 (d, J=6.6 Hz, 6H); ¹³C NMR (100 MHz, CDCl₃): δ (ppm) 171.7, 143.2, 133.6, 129.9, 125.1, 62.3, 53.7, 44.9, 30.1, 22.5, 22.3; ¹⁹F NMR (376 MHz, CDCl₃) δ (ppm) −78.59; HRMS (ESI-TOF) m/z: [M-TfO]⁺ Calcd for C₁₄H₂₂NO₂⁺ 236.1645; Found 236.1642. Isomer B: ¹H NMR (400 MHz, CDCl₃): δ (ppm) 7.32-7.18 (m, 4H), 3.72 (q, J=7.1 Hz, 1H), 3.66 (s, 3H), 3.58 (d, J=7.5 Hz, 1H), 2.72 (brs, 3H), 1.94-1.82 (m, J=6.8 Hz, 1H), 1.49 (d, J=7.2 Hz, 3H), 0.97 (d, J=6.6 Hz, 3H), 0.76 (d, J=6.7 Hz, 3H); ¹³C NMR (100 MHz, CDCl₃): δ (ppm) 175.1, 142.7, 139.3, 127.40, 127.37, 62.1, 52.0, 45.1, 34.9, 19.7, 18.9, 18.6; ¹⁹F NMR (376 MHz, CDCl₃) δ (ppm) −78.48; HRMS (ESI-TOF) m/z: [M-TfO]⁺ Calcd for C₁₄H₂₂NO₂⁺ 236.1645; Found 236.1642.

### Example 65. Synthesis of

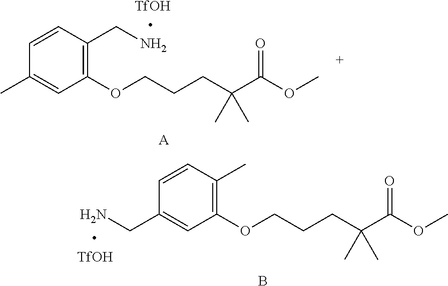

Using general synthetic process 3, the reaction solution was stirred and reacted at 60° C. for 12 h, and after workup, 77.3 mg of primary amine salt was obtained as the regioisomer mixture. Appearance: semi-solid, separation yield: 72% (total separation yield of regioisomers); 2:1 r.r. (A/B). Although the regioisomer mixture could not be completely separated, a portion of pure primary amine product could be obtained by purification over column chromatography. Isomer A: ¹H NMR (400 MHz, CD₃OD): δ (ppm) 7.19 (d, J=7.6 Hz, 1H), 6.86 (s, 1H), 6.80 (d, J=7.6 Hz, 1H), 4.90 (s, 3H, NH₃), 4.12-3.97 (m, 4H), 3.65 (s, 3H), 2.34 (s, 3H), 1.84-1.70 (m, 4H), 1.22 (s, 6H); ¹³C NMR (100 MHz, CD₃OD): δ (ppm) 178.6, 156.9, 141.1, 130.0, 121.0, 120.4 (q, J=318.4 Hz), 118.8, 112.1, 67.8, 51.0, 41.9, 39.0, 36.6, 24.7, 24.2, 20.3; ¹⁹F NMR (376 MHz, CD₃OD) δ (ppm) −80.05; HRMS (ESI-TOF) m/z: [M-TfO]⁺ Calcd for C₁₆H₂₆NO₃⁺ 280.1907; Found 280.1909. Isomer B: 1H NMR (400 MHz, CD₃OD): δ (ppm) 7.13 (d, J=7.5 Hz, 1H), 6.91 (d, J=1.6 Hz, 1H), 6.85 (dd, J=7.5 Hz, 1.6 Hz, 1H), 4.92 (s, 3H, NH₃), 3.98 (t, J=3.0 Hz, 2H), 3.94 (s, 2H), 3.64 (s, 3H), 2.19 (s, 3H), 1.76-1.70 (m, 4H), 1.21 (s, 6H); ¹³C NMR (100 MHz, CD₃OD): δ (ppm) 178.5, 157.5, 131.6, 130.7, 127.5, 120.4 (q, J=318.5 Hz), 120.3, 111.0, 67.8, 50.9, 43.0, 41.8, 36.9, 24.8, 24.2, 14.7; 19F NMR (376 MHz, CD₃OD) δ (ppm) −80.12; HRMS (ESI-TOF) m/z: [M-TfOH+Na]⁺ Calcd for C₁₆H₂₅NNaO₃⁺ 302.1727; Found 302.1732.

### Example 66. Synthesis of

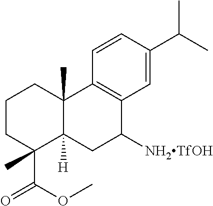

Using general synthetic process 3, the reaction solution was stirred and reacted at 60° C. for 24 h, and after workup, 76.0 mg of primary amine salt was obtained. Appearance: colorless liquid, isolation yield: 63% (total separation yield of diastereomers); dr=3.7:1; Mixture of diastereoisomers: ¹H NMR (400 MHz, CDCl₃): δ (ppm) 7.31 (d, J=2.0 Hz, 0.3H), 7.22 (d, J=2.0 Hz, 1H), 7.20-7.05 (m, 2.6H), 4.55 (brs, 3.8H), 4.34 (d, J=5.1 Hz, 1H), 4.19 (dd, J=10.3 Hz, 7.6 Hz, 0.3H), 3.64 (s, 0.8H), 3.27 (s, 3H), 2.99-2.77 (m, 1.3H), 2.41 (d, J=12.8 Hz, 1H), 2.34-2.23 (m, 1.6H), 2.14 (td, J=13.7 Hz, 5.3 Hz, 1H), 1.89-1.48 (m, 8H), 1.33-0.93 (m, 16H); ¹³C NMR (100 MHz, CDCl₃): δ (ppm) 179.5, 178.9, 147.3, 146.9, 146.8, 146.6, 137.0, 133.6, 127.9, 126.8, 125.5, 125.2, 124.6, 124.4, 120.1 (q, J=318.8 Hz), 52.2, 52.1, 51.7, 50.2, 47.3, 47.0, 43.8, 39.4, 38.1, 37.7, 37.5, 37.4, 37.1, 36.5, 33.7, 33.5, 32.3, 28.6, 25.5, 24.4, 24.0, 23.85, 23.83, 23.79, 18.49, 18.45, 16.4, 16.2; ¹⁹F NMR (376 MHz, CDCl₃) δ (ppm) −78.38; HRMS (ESI-TOF) m/z: [M-TfO]⁺ Calcd for C₂₁H₃₂NO₂⁺ 330.2428; Found 330.2435.

### Example 67. Synthesis of

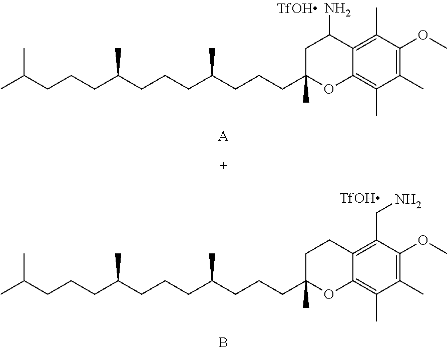

Using general synthetic process 3, the reaction solution was stirred and reacted at 25° C. for 12 h, and after workup, 61.0 mg of primary amine salt was obtained. Appearance: semi-solid, isolation yield: 40% (total separation yield of isomers); 2.6:1 r.r. (A/B); Although the regioisomer mixture could not be completely separated, a portion of pure primary amine product could be obtained by purification over column chromatography. Isomer A: In ¹H NMR, it was observed that compound A was a mixture of diastereomers, but it was difficult to calculate the exact ratio. Therefore, HPLC analysis was used to determine the accurate diastereomer ratio [Daicel chiralpak IC, acetonitrile/water (0.1% phosphoric acid)=45/55, 1.0 mL/min, 2=220 nm, t (main peak)=22.299 min, t (minor peak)=25.112 min); Isomer A: dr=1.3:1; Mixture of diastereoisomers: ¹H NMR (400 MHz, CDCl₃): δ (ppm) 4.20-4.02 (m, 1H), 3.65 (s, 3H), 2.32 (d, J=2.8 Hz, 3H), 2.18 (s, 3H), 2.14-2.04 (m, 4H), 2.01-1.75 (m, 4H), 1.55-1.46 (m, 2H), 1.40-1.06 (m, 22H), 0.89-0.82 (m, 12H); ¹³C NMR (100 MHz, CDCl₃): δ (ppm) 150.5, 150.4, 147.3, 147.1, 129.3, 129.2, 125.95, 125.91, 123.85, 123.80, 123.2, 122.9, 76.1, 75.7, 60.19, 60.17, 43.9, 43.8, 42.70, 42.64, 42.21, 42.15, 41.64, 41.61, 40.6, 39.4, 37.6, 37.5, 37.43, 37.40, 37.38, 37.33, 37.31, 32.8, 32.73, 32.69, 32.67, 32.64, 27.9, 25.7, 24.83, 24.82, 24.48, 24.44, 23.7, 22.74, 22.65, 21.3, 21.1, 19.8, 19.72, 19.70, 19.64, 19.58, 12.74, 12.70, 12.4, 11.98, 11.95; ¹⁹F NMR (376 MHz, CDCl₃) δ (ppm) −78.34; HRMS (ESI-TOF) m/z: [M-TfOH+Na]⁺ Calcd for C₃₀H₅₃NNaO₂⁺ 482.3969; Found 482.3966. Isomer B: ¹H NMR (400 MHz, CDCl₃): δ (ppm) 4.08 (s, 2H), 3.61 (s, 3H), 3.54 (brs, 3H), 2.56 (t, J=6.7 Hz, 2H), 2.27 (s, 3H), 2.15 (s, 3H), 1.92-1.74 (m, 2H), 1.61-1.47 (m, 3H), 1.37-1.09 (m, 21H), 0.88-0.80 (m, 12H); ¹³C NMR (100 MHz, CDCl₃): δ (ppm) 150.0, 148.2, 130.3, 128.5, 119.5, 118.7, 60.4, 39.5, 39.4, 37.8, 37.47, 37.44, 37.40, 37.3, 32.8, 32.70, 32.67, 28.0, 24.8, 24.5, 24.0, 22.7, 22.6, 21.4, 20.3, 19.73, 19.66, 19.56, 19.50, 12.0, 11.8; ¹⁹F NMR (376 MHz, CDCl₃) δ (ppm) −78.38; HRMS (ESI-TOF) m/z: [M-TfOH+Na]⁺ Calcd for C₃₀H₅₃NNaO₂⁺ 482.3969; Found 482.3970.

### Example 68. Synthesis of

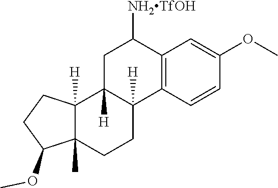

General synthetic process 3 was used. To a dry round-bottom flask, were successively added the supported catalyst iron octachlorophthalocyanine (5 mol %, supported on 1 g of silica gel), compound 1 (0.25 mmol), dioxane (3 mL), and the aqueous solution of nitrogen source 2 (0.75 mmol) (7 mL). The reaction solution was stirred at 100° C. for 24 h, and after workup, 65.3 mg of primary amine salt was obtained. Appearance: semi-solid, isolation yield: 56% (total isolation yield of diastereomers); dr=1.2:1; Mixture of diastereoisomers: ¹H NMR (400 MHz, CDCl₃): δ (ppm) 7.25-7.12 (m, 1H), 6.99-6.85 (m, 1H), 6.84-6.72 (m, 1H), 4.77 (s, 3H, NH₃), 4.28-4.16 (m, 1H), 3.74 (s, 3H), 3.36 (s, 3H), 3.33-3.24 (m, 1H), 2.33-1.91 (m, 6H), 1.57-1.25 (m, 7H), 0.83-0.70 (m, 3H); ¹³C NMR (100 MHz, CDCl₃): δ (ppm) 158.12, 158.06, 136.9, 135.8, 132.9, 132.5, 126.8, 126.7, 119.9 (q, J=318.6 Hz), 115.0, 114.2, 113.5, 111.5, 90.6, 57.89, 57.86, 55.31, 55.30, 50.8, 49.7, 49.5, 49.4, 43.8, 43.7, 43.4, 43.0, 37.8, 37.7, 35.8, 33.7, 32.9, 27.7, 26.3, 25.9, 22.8, 22.6, 11.5, 11.4; 19F NMR (376 MHz, CDCl₃) δ (ppm) −78.38; HRMS (ESI-TOF) m/z: [M-TfOH+Na]⁺ Calcd for C₂₀H₂₉NNaO₂⁺ 338.2091; Found 338.2094.

### Example 69. Synthesis of

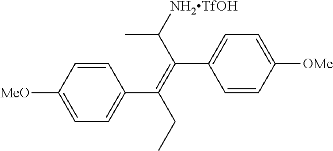

Using general synthetic process 3, the reaction solution was stirred and reacted at 100° C. for 12 h, and after workup, 34.6 mg of primary amine salt was obtained. Appearance: colorless liquid, isolation yield: 30%; ¹H NMR (400 MHz, CDCl₃): δ (ppm) 7.23-7.01 (m, 4H), 6.99-6.81 (m, 4H), 4.85 (brs, 3H), 3.99 (q, J=6.7 Hz, 1H), 3.82 (s, 3H), 3.81 (s, 3H), 2.00 (q, J=7.5 Hz, 2H), 1.10 (d, J=6.7 Hz, 3H), 0.71 (t, J=7.5 Hz, 3H); ¹³C NMR (100 MHz, CDCl₃): δ (ppm) 159.0, 158.6, 145.2, 135.2, 132.6, 131.3, 129.4, 127.9, 114.1, 113.9, 55.21, 55.19, 49.0, 29.2, 19.9, 12.5; ¹⁹F NMR (376 MHz, CDCl₃) δ (ppm) −78.46; HRMS (ESI-TOF) m/z: [M-TfO]⁺ Calcd for C₂₀H₂₆NO₂⁺ 312.1958; Found 312.1949.

### Example 70. Synthesis of

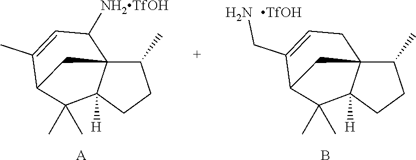

Using general synthetic process 3, the reaction solution was stirred and reacted at 25° C. for 12 h, and after workup, 68.3 mg of primary amine salt was obtained. Appearance: white solid, separation yield: 74% (total separation yield of regioisomers); 2:1 r.r. (A/B). Although the regioisomer mixture could not be completely separated, a portion of pure primary amine product could be obtained by purification over column chromatography. Isomer A: white solid; m.p. 185-187° C.; ¹H NMR (400 MHz, CDCl₃): δ (ppm) 7.05 (brs, 3H, NH₃), 5.44-5.31 (m, 1H), 3.63 (s, 1H), 2.14-2.03 (m, 1H), 1.99-1.79 (m, 3H), 1.75 (s, 3H), 1.73-1.64 (m, 2H), 1.61-1.41 (m, 3H), 0.99 (s, 3H), 0.98-0.88 (m, 6H); ¹³C NMR (100 MHz, CDCl₃): δ (ppm) 149.3, 119.9 (d, J=318.6 Hz), 115.4, 57.5, 55.6, 53.8, 53.5, 46.3, 36.5, 34.0, 33.9, 27.8, 25.5, 24.6, 23.3, 14.7; ¹⁹F NMR (376 MHz, CDCl₃) δ (ppm) −78.41; HRMS (ESI-TOF) m/z: [M-TfO]⁺ Calcd for C₁₅H₂₆N⁺ 220.2060; Found 220.2070. Isomer B: yellow liquid; ¹H NMR (400 MHz, CDCl₃): δ (ppm) 6.86 (brs, 3H, NH₃), 5.65 (t, J=3.4 Hz, 1H), 3.60-3.31 (m, 2H), 2.23 (d, J=17.5 Hz, 1H), 1.99-1.51 (m, 7H), 1.50-1.28 (m, 3H), 0.97 (d, J=6.3 Hz, 6H), 0.84 (d, J=7.1 Hz, 3H); ¹³C NMR (100 MHz, CDCl₃): δ (ppm) 135.7, 126.6, 58.9, 53.7, 51.2, 48.7, 45.7, 41.3, 40.0, 38.6, 36.0, 27.2, 25.1, 24.8, 15.3; ¹⁹F NMR (376 MHz, CDCl₃) δ (ppm) −78.19; HRMS (ESI-TOF) m/z: [M-TfO]⁺ Calcd for C₁₅H₂₆N⁺ 220.2060; Found 220.2070.

### Example 71. Synthesis of

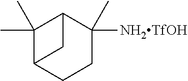

Using general synthetic process 3, the reaction solution was stirred and reacted at 25° C. for 12 h, and after workup, 71.0 mg of primary amine salt was obtained. Appearance: white solid, isolation yield: 94%; m.p.: 98-100° C.; ¹H NMR (400 MHz, CD₃OD): δ (ppm) 4.94 (brs, 3H), 2.44-2.31 (m, 1H), 2.09-1.78 (m, 6H), 1.43 (s, 3H), 1.35 (d, J=11.0 Hz, 1H), 1.32 (s, 3H), 1.05 (s, 3H); ¹³C NMR (100 MHz, CD₃OD): δ (ppm) 120.4 (q, J=318.5 Hz), 58.9, 50.9, 39.9, 38.6, 27.1, 26.5, 26.4, 26.3, 23.9, 22.5; ¹⁹F NMR (376 MHz, CD₃OD) δ (ppm) −80.05; HRMS (ESI-TOF) m/z: [M-TfO]⁺ Calcd for C₁₀H₂₀N⁺ 154.1590; Found 154.1583.

### Example 72. Synthesis of

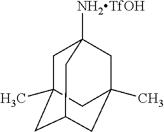

Using general synthetic process I, the reaction solution was stirred and reacted at 25° C. for 12 h, and after workup, 104.1 mg of primary amine salt was obtained. Appearance: white solid; isolation yield: 63%. M.p. 222-224° C.; ¹H NMR (400 MHz, CD₃OD): δ (ppm) 4.87 (s, 3H, NH₃), 2.30-2.19 (m, 1H), 1.78-1.63 (m, 2H), 1.59-1.37 (m, 8H), 1.28-1.14 (m, 2H), 0.92 (s, 6H); ¹³C NMR (100 MHz, CD₃OD): δ (ppm) 120.4 (q, J=318.3 Hz), 53.0, 49.4, 46.0, 41.4, 38.6, 32.1, 29.8, 28.7; ¹⁹F NMR (376 MHz, CD₃OD): δ (ppm) −80.04; HRMS (ESI-TOF) m/z: [M-TfO]⁺ Calcd for C₁₂H₂₂N⁺ 180.1747; Found 180.1753.

Preparation on a gram scale for Example 72: To a dry round-bottom flask, were sequentially added the supported catalyst iron octachlorophthalocyanine (5 mol %, supported on 32 g of silica gel), compound 1 (8.0 mmol), dioxane (32.0 mL), and the aqueous solution of nitrogen source 2 (24.0 mmol) (608.0 mL). The reaction solution was stirred and reacted at 25° C. for 24 h, and the reaction was detected by GC-MS until compound 1 was completely consumed, resulting in the formation of primary amine products. After completion of the reaction, the reaction solution was filtered, and the catalyst in the filter cake was rinsed with water. The filtrates were combined and extracted with ethyl acetate (100 mL) to remove organic impurities. The aqueous layer was concentrated to dry by rotatory evaporation under reduced pressure, and the residue was purified by column chromatography to obtain 1.54 g of primary amine salt product. Appearance: white solid, isolation yield: 58%.

### General Synthetic Process 4

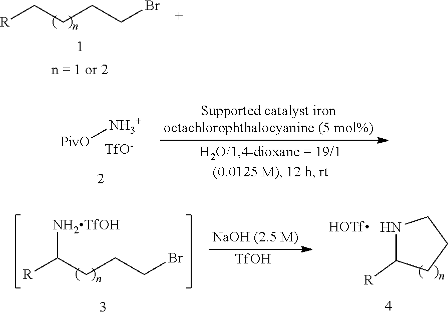

To a dry round-bottom flask, were added the supported catalyst iron octachlorophthalocyanine (5 mol %, supported on 2 g of silica gel), compound 1 (0.5 mmol), dioxane (2 mL), and the aqueous solution of nitrogen source 2 (1.5 mmol) (38 mL) in sequence. The reaction solution was stirred at 25° C. for 12 h, and the reaction was monitored by TLC until compound 1 was completely consumed, resulting in the formation of primary amine product 3. After completion of the reaction, 5 mL of aqueous NaOH solution (2.5 mol/L) was added to promote the cyclization reaction, and the reaction was detected by TLC until the primary amine product was completely converted to the cyclization product. Trifluoromethanesulfonic acid (2.4 mL) was added in an ice-bath until pH=1.5, and the resultant solution was stirred and reacted at 25° C. for 1 h. Then, the solvent was rotatory evaporated to dry under reduced pressure, and the residue was purified by column chromatography to obtain the cyclization product 4.

### Example 73. Synthesis of

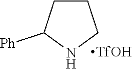

Using general synthetic process 4, the reaction solution was stirred and reacted at 25° C. for 12 h. After workup, 96.6 mg of cyclization product was obtained. Appearance: brown liquid, separation yield: 65%; ¹H NMR (400 MHz, CD₃OD): δ (ppm) 7.63-7.29 (m, 5H), 4.89 (s, 2H, NH₂), 4.75-4.53 (m, 1H), 3.56-3.37 (m, 2H), 2.56-2.40 (m, 1H), 2.40-2.07 (m, 3H); ¹³C NMR (100 MHz, CD₃OD): δ (ppm) 134.5, 129.2, 129.0, 127.2, 120.4 (q, J=318.5 Hz), 63.3, 45.1, 30.3, 23.4; ¹⁹F NMR (376 MHz, CD₃OD): δ (ppm) −80.08; HRMS (ESI-TOF) m/z: [M-TfO]⁺ Calcd for C₁₀H₁₄N⁺ 148.1121; Found 148.1129.

### Example 74. Synthesis of

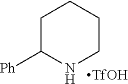

Using general synthetic process 4, the reaction solution was stirred and reacted at 25° C. for 12 h. After workup, 116.7 mg of cyclization product was obtained. Appearance: colorless liquid, separation yield: 75%; ¹H NMR (400 MHz, CD₃OD): δ (ppm) 7.58-7.30 (m, 5H), 4.98 (brs, 2H, NH₂), 4.24 (dd, J=11.6 Hz, 3.2 Hz, 1H), 3.52-3.42 (m, 1H), 3.27-3.04 (m, 1H), 2.16-1.89 (m, 4H), 1.89-1.64 (m, 2H); ¹³C NMR (100 MHz, CD₃OD): δ (ppm) 136.9, 129.1, 128.9, 126.8, 60.7, 45.4, 29.9, 22.4, 21.7; ¹⁹F NMR (376 MHz, CD₃OD) δ (ppm) −80.01; HRMS (ESI-TOF) m/z: [M-TfO]⁺ Calcd for C₁₁H₁₆N⁺ 162.1277; Found 162.1286.

### General Synthetic Process 5

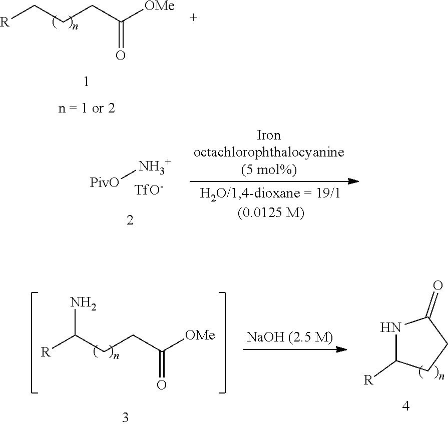

To a dry round-bottom flask, were added the catalyst iron octachlorophthalocyanine (5 mol %), compound 1 (0.5 mmol), dioxane (2 mL), and the aqueous solution of nitrogen source 2 (1.5 mmol) (38 mL) in sequence. The reaction solution was stirred and reacted at 20-100° C. for 12-36 h, and the reaction was monitored via TLC until compound 1 was completely consumed, resulting in the formation of primary amine product 3. After completion of the reaction, 5 mL of NaOH (2.5 mol/L) aqueous solution was added to promote the cyclization reaction, and the reaction was detected via TLC until the primary amine product was completely converted to the cyclization product. Then, the aqueous phase was extracted with ethyl acetate. The organic phase was washed with saturated saline. The solvent was rotatory evaporated to dry under reduced pressure, and the residue was purified by column chromatography to obtain cyclization product 4.

### Example 75. Synthesis of

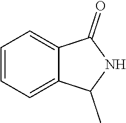

Using general synthetic process 5, the reaction solution was stirred and reacted at 60° C. for 36 h. After workup, 55.2 mg of cyclization product was obtained. Appearance: yellow solid, isolation yield: 75%; melting point: 152-154° C.; ¹H NMR (400 MHz, CDCl₃): δ (ppm) 8.02 (brs, 1H), 7.83 (d, J=7.5 Hz, 1H), 7.61-7.50 (m, 1H), 7.50-7.36 (m, 2H), 4.70 (q, J=6.7 Hz, 1H), 1.50 (d, J=6.7 Hz, 3H); ¹³C NMR (100 MHz, CDCl₃): δ (ppm) 171.2, 149.0, 131.9, 131.7, 128.0, 123.7, 122.2, 52.7, 20.3; HRMS (ESI-TOF) m/z: [M+Na]⁺ Calcd for CH₉NNaO⁺ 170.0576; Found 170.0583.

### Example 76. Synthesis of

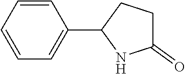

Using general synthetic process 5, the reaction solution was stirred and reacted at 50° C. for 16 h. After workup, 49.1 mg of cyclization product was obtained. Appearance: white solid, isolation yield: 61%; melting point: 68-70° C.; ¹H NMR (400 MHz, CDCl₃): δ (ppm) 7.50-7.15 (m, 5H), 6.11 (brs, 1H), 4.78 (t, J=7.2 Hz, 1H), 2.73-2.31 (m, 3H), 2.10-1.91 (m, 1H); ¹³C NMR (100 MHz, CDCl₃): δ (ppm) 179.0, 142.5, 129.0, 128.0, 125.7, 58.1, 31.5, 30.4; HRMS (ESI-TOF) m/z: [M+Na]⁺ Calcd for C₁₀H₁₁NNaO⁺ 184.0733; Found 184.0732.

### Example 77. Synthesis of

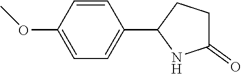

Using general synthetic process 5, the reaction solution was stirred and reacted at 60° C. for 36 h. After workup, 53.5 mg of cyclization product was obtained. Appearance: white solid, isolation yield: 56%; melting point: 123-125° C.; ¹H NMR (400 MHz, CDCl₃): δ (ppm) 7.19-7.08 (m, 2H), 6.88-6.73 (m, 2H), 6.70 (brs, 1H), 4.62 (t, J=7.1 Hz, 1H), 3.71 (s, 3H), 2.49-2.23 (m, 3H), 1.92-1.76 (m, 1H); ¹³C NMR (100 MHz, CDCl₃): δ (ppm) 178.7, 159.2, 134.6, 126.9, 114.2, 57.7, 55.3, 31.5, 30.5; HRMS (ESI-TOF) m/z: [M+Na]⁺ Calcd for C₁₁H₁₃NNaO₂⁺ 214.0838; Found 214.0844.

### Example 78. Synthesis of

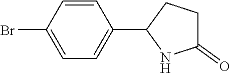

Using general synthetic process 5, the reaction solution was stirred and reacted at 60° C. for 36 h. After workup, 46.0 mg of cyclization product was obtained. Appearance: white solid, isolation yield: 38%; melting point: 138-140° C.; ¹H NMR (400 MHz, CDCl₃): δ (ppm) 7.56-7.42 (m, 2H), 7.23-7.09 (m, 2H), 6.97 (brs, 1H), 4.73 (t, J=7.1 Hz, 1H), 2.64-2.48 (m, 1H), 2.48-2.27 (m, 2H), 2.01-1.75 (m, 1H); ¹³C NMR (100 MHz, CDCl₃): δ (ppm) 178.9, 141.6, 132.0, 127.4, 121.7, 57.6, 31.2, 30.4; HRMS (ESI-TOF) m/z: [M+Na]⁺ Calcd for C₁₀H₁₀⁷⁹BrNNaO⁺ 261.9838; Found 261.9829; Calcd for C₁₀H₁₀⁸¹BrNNaO⁺ 263.9818; Found 263.9810.

### Example 79. Synthesis of

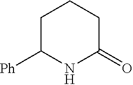

Using general synthetic process 5, the reaction solution was stirred and reacted at 50° C. for 16 h. After workup, 38.5 mg of cyclization product was obtained. Appearance: white solid, isolation yield: 44%; melting point: 131-135° C.; ¹H NMR (400 MHz, CDCl₃): δ (ppm) 7.48-7.20 (m, 5H), 6.08 (brs, 1H), 4.57 (dd, J=9.1 Hz, 4.6 Hz, 1H), 2.58-2.36 (m, 2H), 2.22-2.01 (m, 1H), 1.98-1.86 (m, 1H), 1.86-1.74 (m, 1H), 1.74-1.61 (m, 1H); ¹³C NMR (100 MHz, CDCl₃): δ (ppm) 172.4, 142.6, 128.8, 127.9, 126.1, 57.7, 32.2, 31.3, 19.7; HRMS (ESI-TOF) m/z: [M+Na]⁺ Calcd for C₁₁H₁₃NNaO⁺ 198.0889; Found 198.0899.

The beneficial effects of the present invention were demonstrated by the following experimental examples.

### Experimental Example 1: Screening of Reaction Catalyst Types and Catalytic Reaction Conditions for the Present Invention

### 1. Selection of Catalyst Types

Preparation of the Product According to the Following Reaction Scheme:

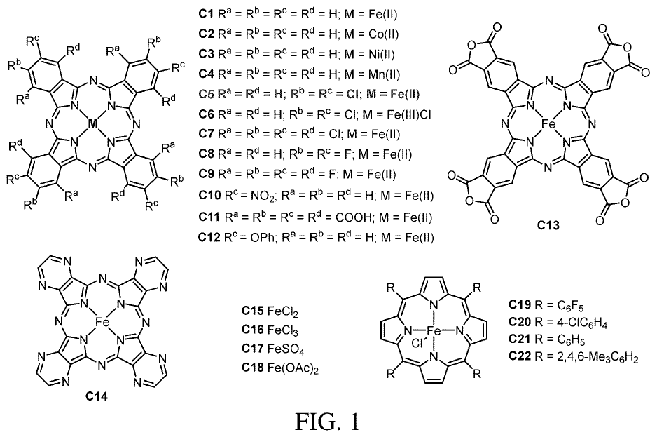

To a dry round-bottom flask, were successively added different catalysts C1-C22 (10 mol %), compound 1a (0.1 mmol), and the aqueous solution of nitrogen source 2 (0.2 mmol) (2 mL), as shown in FIG. 1. The reaction solution was stirred and reacted at room temperature for 12 h, and the reaction was monitored via TLC or GC-MS until compound 1a was completely consumed, resulting in the formation of primary amine product. Then, the solvent was rotatory evaporated to dry under reduced pressure, and the residue was purified by column chromatography, to obtain the primary amine product 3a.

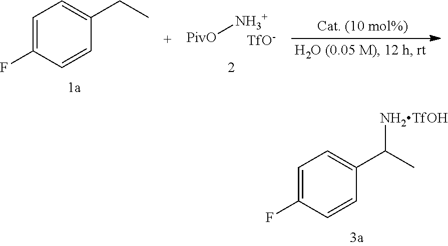

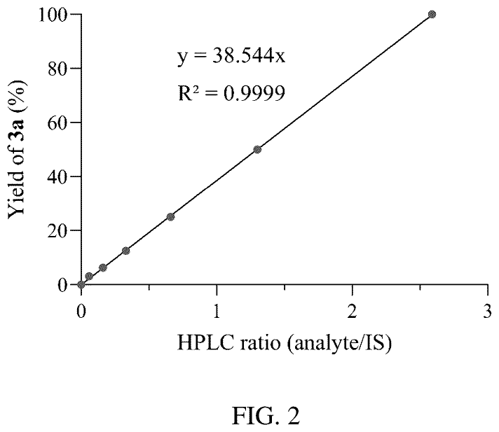

A UV quantitative analysis method was established using HPLC, and a standard curve was plotted for calculating the analytical yield of the product (as shown in FIG. 2). After completion of the reaction, the reaction solution was analyzed using HPLC, so as to calculate the UV absorption ratio of the primary amine product to the internal standard (1,3,5-trimethoxybenzene) at 260 nm. Then, the analytical yield of the primary amine product catalyzed by different catalysts was determined based on the standard curve.

TABLE 1
Isolated yield of primary amine products from catalytic
reactions catalyzed by different catalysts.

| | | Isolated |
|---|---|---|
| No. | Catalyst | yield (%) |
| 1 | C1 | 1 |
| 2 | C2 | <1 |
| 3 | C3 | <1 |
| 4 | C4 | <1 |
| 5 | C5 | 32 |
| 6 | C6 | 31 |
| 7 | C7 | 10 |
| 8 | C8 | 10 |
| 9 | C9 | 10 |
| 10 | C10 | 1 |
| 11 | C11 | <1 |
| 12 | C12 | <1 |
| 13 | C13 | <1 |
| 14 | C14 | <1 |
| 15 | C15 | 0 |
| 16 | C16 | 0 |
| 17 | C17 | 0 |
| 18 | C18 | 0 |
| 19 | C19 | 1 |
| 20 | C20 | <1 |
| 21 | C21 | <1 |
| 22 | C22 | <1 |

The results are presented in Table 1. As shown, catalysts C5 to C9 all exhibit good catalytic effects, with catalyst C5 (iron octachlorophthalocyanine) being the most preferred.

### 2. Screening of Reaction Solvent Types

Based on the above catalytic reaction conditions, C5 (iron octachlorophthalocyanine) was used as the catalyst to continue the screening of reaction solvent types, as shown in Table 2.

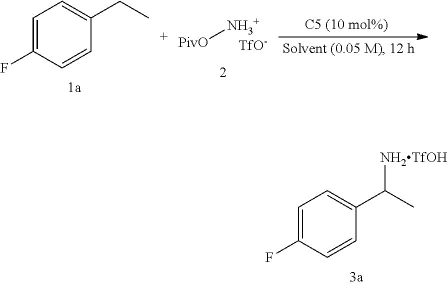

TABLE 2
Isolated yield of primary amine products obtained
using different reaction solvents.

| | | Isolated |
|---|---|---|
| No. | Reaction solvents | yield (%) |
| 1 | H₂O | 32 |
| 2 | MeCN | <1 |
| 3 | 1,4-dioxane | 0 |
| 4 | HFIP | <1 |
| 5 | MeOH | 3 |
| 6 | THF | <1 |
| 7 | DMF | 0 |
| 8 | THF/H₂O = 2/8 | 2 |
| 9 | DMF/H₂O = 2/8 | 34 |
| 10 | MeCN/H₂O = 2/8 | 14 |
| 11 | 1,4-dioxane/H₂O = 2/8 | 34 |
| 12 | 1,4-dioxane/H₂O = 1/19 | 36 |
| 13 | 1,4-dioxane/H₂O = 1/9 | 34 |
| 14 | 1,4-dioxane/H₂O = 4/6 | 26 |
| 15^{c} | 1,4-dioxane/H₂O = 1/19 | 35 |
| 16^{d} | 1,4-dioxane/H₂O = 1/19 | 31 |

The results are presented in Table 2. As shown, using water, a mixed solution of N,N-dimethylformamide (DMF) and water, a mixed solution of acetonitrile (MeCN) and water, or a mixed solution of 1,4-dioxane and water as the reaction solvent, good catalytic effects were obtained. Among them, the solution resulted from mixing 1,4-dioxane and water in a ratio of 1:19 was the most preferred.

### 3. Screening for the Amount of Catalyst C5 and the Concentration of Substrate Reaction

Based on the above catalytic reaction conditions, a solution obtained by mixing 1,4-dioxane and water in a ratio of 1:19 was used as the reaction solvent, to perform the screening for the amount of catalyst C5 as a mole percentage of compound 1a and for the concentration of compound 1a, as shown in Table 3.

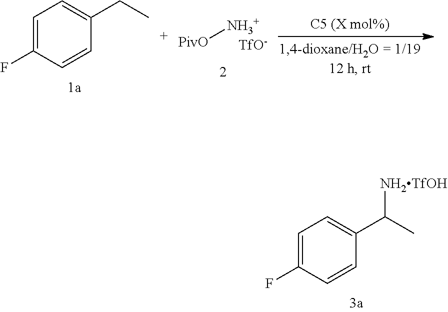

TABLE 3
Isolated yield of primary amine products obtained
using different mole percentages of catalyst C5
and different concentrations of compound 1a.

| | | Concentrations of | Isolated |
|---|---|---|---|
| No. | Mole percentage of C5 (%) | compound 1a | yield (%) |
| 1 | 10 | 0.05M | 36 |
| 2 | 15 | 0.05M | 34 |
| 3 | 2 | 0.05M | 22 |
| 4 | 4 | 0.05M | 29 |
| 5 | 5 | 0.05M | 37 |
| 6^{c} | 5 | 0.05M | 34 |
| 7 | 5 | 0.025M | 38 |
| 8 | 5 | 0.0125M | 40 |
| 9 | 5 | 0.00625M | 32 |

The results are presented in Table 3. As shown, under the conditions of the mole percentage of catalyst C5 being 2 mol % to 10 mol % as well as the concentration of compound 1a being 0.00625 M to 0.05 M, good catalytic effects were obtained. The most preferable is the mole percentage of catalyst C5 being 5 mol % and the concentration of compound 1a being 0.0125 M.

### 4. Screening for the Types and Equivalents of Nitrogen Source 2

Based on the above catalytic reaction conditions, the mole percentage of catalyst C5 was set at 5 mol %, and the concentration of compound 1a was set at 0.0125 M, so as to carry out the screening for the types and equivalents (based on the amount of compound 1a as 1 equivalent) of nitrogen source 2, as shown in Table 4.

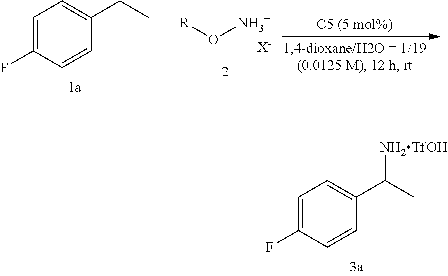

TABLE 4
Isolated yield of primary amine products obtained by using
different types and equivalents of nitrogen source 2.

| | | Equivalent of | Isolated |
|---|---|---|---|
| No. | Nitrogen source (2) | nitrogen source | yield (%) |
| 1 | PivONH₂•TfOH | 2 | 40 |
| 2 | AcONH₂•TfOH | 2 | 33 |
| 3 | PivONH₂•HCl | 2 | 15 |
| 4 | NH₂OH•HCl | 2 | <1 |
| 5 | MsONH₂•TfOH | 2 | <1 |
| 6 | TsONH₂•TfOH | 2 | 0 |
| 7 | NO₂ONH₂•TfOH | 2 | 0 |
| 8 | CH₃ONH₂•TfOH | 2 | 0 |
| 9 | PivONH₂•TfOH | 1 | 27 |
| 10 | PivONH₂•TfOH | 1.5 | 29 |
| 11 | PivONH₂•TfOH | 3 | 46 (Isolated |
| | | | yield 68%) |
| 12 (Note) | PivONH₂•TfOH | 3 | 50 (Isolated |
| | | | yield 76%) |

(Note:
Using supported catalyst C5)

The results are presented in Table 4. It was evident that using PivONH₂·TfOH, AcONH₂·TfOH, and PivONH₂·HCl as nitrogen sources, and controlling the equivalent of nitrogen source to be 1-3, good catalytic effects were obtained. Among them, using PivONH₂·TfOH as the nitrogen source and controlling the equivalent nitrogen source to be 3 is the most preferable.

In summary, the present invention provided a catalyst suitable for primary amination of sp³-carbon-hydrogen bonds, as well as a method for preparing primary amine compounds using this catalyst to catalyze the primary amination of sp³-carbon-hydrogen bonds. The method of the present invention was highly versatile, with a wide range of substrate applicability, environmentally friendly and mild reaction conditions that are not harsh. The reaction operation was simple, and thus the method had potential industrial application prospects.

1. The use of the compound represented by formula I as a catalyst for primary amination of sp³-carbon-hydrogen bonds: 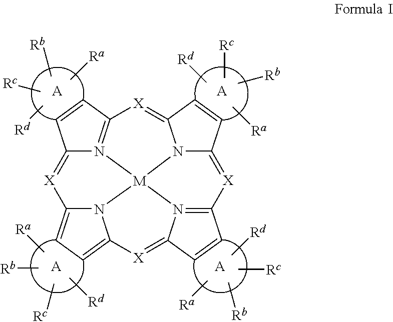
wherein, fused ring A is selected from the group consisting of 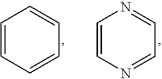
or absence; R^{a}, R^{b}, R^{c}, and R^{d} are each independently selected from the group consisting of H, halogen, alkyl, alkoxy, nitro, carboxylic acid, ester group, and alternatively, any two of R^{a}, R^{b}, R^{c}, and R^{d} are linked to form a ring; X is N or CR^{e}, and R^{e} is selected from the group consisting of aromatic rings or substituted aromatic rings or aromatic heterocycles with any group; Mis a metal ion.

2. The use according to claim 1, characterized in that R^{a}, R^{b}, R^{c}, and R^{d} are each independently selected from the group consisting of H and halogens, and at least one of them is halogen.

3. The use according to claim 1, characterized in that said M is selected from the group consisting of Fe²⁺, Fe³⁺, Co²⁺, Ni²⁺, or Mn²⁺.

4. The use according to claim 3, characterized in that said Mis Fe²⁺.

5. The use according to claim 1, characterized in that said fused ring A represents 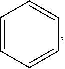
X represents nitrogen, and the halogen represents chlorine or fluorine.

6. The use according to claim 5, characterized in that R^{a} and R^{d} are hydrogen, while R^{b} and R^{c} are chlorine.

7. The use according to claim 1, characterized in that the compound represented by formula I is iron octachlorophthalocyanine.

8. The use according to any one of claims 1 to 7, characterized in that the primary amination of sp³-carbon-hydrogen bond involves the use of compound 1 and compound 2 as reactants, which are catalyzed by a catalyst to produce compound 3, and the reaction scheme is as follows: 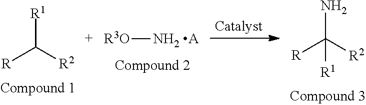
wherein, R, R¹, and R² are each independently selected from any groups, and alternatively, any two or three of R, R¹, and R² are linked to form a substituted or unsubstituted ring; R³ is selected from the group consisting of H, pivaloyl, acetyl, methanesulfonyl, p-toluenesulfonyl, sulfonic acid group, nitro, or methyl; A is selected from the group consisting of sulfonic acid, hydrochloric acid, sulfuric acid, acetic acid, or absence.

9. The use according to claim 8, characterized in that R³ represents pivaloyl; A represents sulfonic acid, and preferably trifluoromethanesulfonic acid.

10. The use according to claim 8, characterized in that the reaction conditions are: reacting in a solvent at 20-100° C. for 12-120 h;
and/or, the molar ratio of compound 1 to compound 2 is 1:(1-3), the molar ratio of compound 1 to the catalyst is 100:(2-10), and the concentration of compound 1 is 0.00625 M to 0.05 M.

11. The use according to claim 10, characterized in that the reaction temperature is 25° C., and the reaction time is 12 h;
and/or, the molar ratio of compound 1 to compound 2 is 1:3, the molar ratio of compound 1 to the catalyst is 100:5, and the concentration of compound 1 is 0.0125 M.

12. The use according to claim 10, characterized in that the solvent is selected from the group consisting of acetonitrile, dioxane, water, hexafluoroisopropanol, dichloromethane, N,N-dimethylformamide, and a mixture thereof.

13. The use according to claim 12, characterized in that the solvent is a mixed solvent of dioxane and water.

14. A method for preparing primary amine compounds, characterized in that the method comprises the step of preparing primary amine compound 3 by reacting compound 1 and compound 2 as reactants under the action of a catalyst; the reaction scheme is as follows: 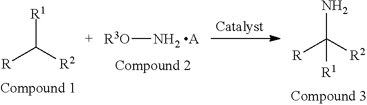
wherein, R, R¹, and R² are each independently selected from any groups, and alternatively, any two or three of R, R¹, and R² are linked to form a substituted or unsubstituted ring; R³ is selected from the group consisting of H, pivaloyl, acetyl, methanesulfonyl, p-toluenesulfonyl, sulfonic acid group, nitro, or methyl; A is selected from the group consisting of sulfonic acid, hydrochloric acid, sulfuric acid, acetic acid, or absence;
the catalyst is a compound represented by formula I: 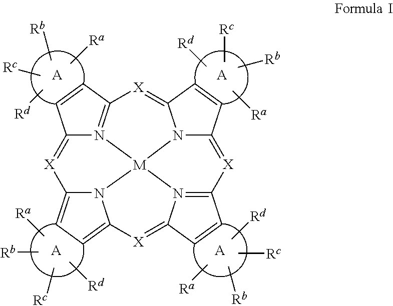
wherein, fused ring A is selected from the group consisting of 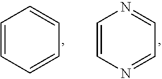
or absence; R^{a}, R^{b}, R^{c}, and R^{d} are each independently selected from the group consisting of H, halogen, alkyl, alkoxy, nitro, carboxylic acid, ester group, and alternatively, any two of R^{a}, R^{b}, R^{c}, and R^{d} are linked to form a ring; X is N or CR^{e}, and R^{e} is selected from the group consisting of aromatic rings or substituted aromatic rings or aromatic heterocycles with any group; M is a metal ion.

15. The method according to claim 14, characterized in that R^{a}, R^{b}, R^{c}, and R^{d} are each independently selected from the group consisting of H and halogens, and at least one of them is halogen.

16. The method according to claim 14, characterized in that said M is selected from the group consisting of Fe²⁺, Fe³⁺, Co²⁺, Ni²⁺, or Mn²⁺.

17. The method according to claim 16, characterized in that said M is Fe²⁺.

18. The method according to claim 14, characterized in that said fused ring A represents 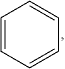
X represents nitrogen, and the halogen represents chlorine or fluorine.

19. The method according to claim 18, characterized in that R^{a} and R^{d} are hydrogen, while R^{b} and R^{c} are chlorine.

20. The method according to claim 14, characterized in that the compound represented by formula I is iron octachlorophthalocyanine.

21. The method according to claim 14, characterized in that R³ represents pivaloyl; A represents sulfonic acid, and preferably trifluoromethanesulfonic acid.

22. The method according to claim 14, characterized in that the reaction conditions are: reacting in a solvent at 20-100° C. for 12-120 h;
and/or, the molar ratio of compound 1 to compound 2 is 1:(1-3), the molar ratio of compound 1 to the catalyst is 100:(2-10), and the concentration of compound 1 is 0.00625 M to 0.05 M.

23. The method according to claim 22, characterized in that the reaction temperature is 25° C., and the reaction time is 12 h;
and/or, the molar ratio of compound 1 to compound 2 is 1:3, the molar ratio of compound 1 to the catalyst is 100:5, and the concentration of compound 1 is 0.0125 M.

24. The method according to claim 22, characterized in that the solvent is selected from the group consisting of acetonitrile, dioxane, water, hexafluoroisopropanol, dichloromethane, N,N-dimethylformamide, and a mixture thereof.

25. The method according to claim 24, characterized in that the solvent is a mixed solvent of dioxane and water.

26. The method according to any one of claims 14 to 25, characterized in that R is a substituted or unsubstituted (5-6)-membered aromatic ring, (5-6)-membered aromatic heterocycle, (5-6)-membered fused (5-6)-membered aromatic ring, or (5-6)-membered fused (5-6)-membered aromatic heterocycle.

27. The method according to claim 26, characterized in that R¹ and R² are each independently selected from any groups, and the reaction scheme is as follows: 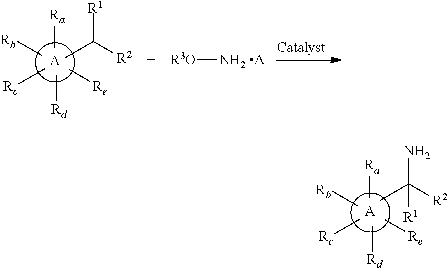
wherein, Rₐ, R_{b}, R_{c}, R_{d}, and Rₑ are each independently selected from H or any group other than H; ring A is selected from the group consisting of

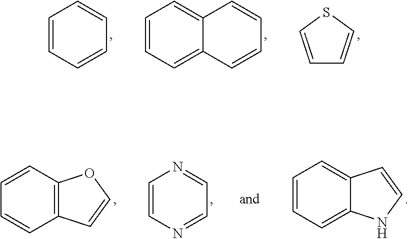

28. The method according to claim 27, characterized in that said Rₐ, R_{b}, R_{c}, R_{d}, and Rₑ are each independently selected from the group consisting of H, halogen, phenyl substituted with straight or branched C₁₋₁₀ alkyl, phenyl, straight or branched C₁₋₁₀ alkyl, straight or branched C₁₋₁₀ alkoxy; and/or, said R¹ and R² are each independently selected from H, (3-6)-membered saturated cycloalkyl, straight or branched C₁₋₁₀ alkyl, phenyl, phenyl-substituted C₁₋₁₀ straight or branched alkyl, or R₁ and R² are linked to form a ring.

29. The method according to claim 27, characterized in that the reaction conditions are: reacting in a mixed solvent of dioxane and water at 20-80° C. for 12-120 h, preferably at 25-70° C. for 12-120 h, and more preferably at 25-60° C. for 12-120 h.

31. The method according to claim 26, characterized in that R¹ is H, R² and R are linked to form a ring, and the reaction scheme is as follows: 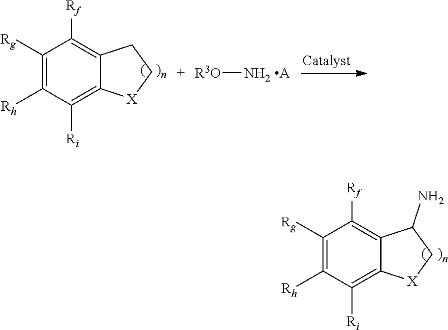
wherein, R_{f}, R_{g}, Rₕ, and Rᵢ are each independently selected from H or any group other than H; X is selected from the group consisting of CH₂, O, or NR′, and n is any integer from 0 to 6; R′ is selected from the group consisting of H, C₁₋₁₈ alkyl, benzyl, or amino-protecting group, and is preferably selected from the group consisting of H, methyl, ethyl, benzyl, benzoyl, or Boc.

31. The method according to claim 30, characterized in that R_{f}, R_{g}, Rₕ, and Rᵢ are each independently selected from the group consisting of H, halogen, phenyl substituted with straight or branched C₁₋₁₀ alkyl, phenyl, straight or branched C₁₋₁₀ alkyl, straight or branched C₁₋₁₀ alkoxy; alternatively, two adjacent groups of R_{f}, R_{g}, Rₕ, and Rᵢ are linked to form a ring.

32. The method according to claim 30, characterized in that the reaction conditions are: reacting in a mixed solvent of dioxane and water at 25-60° C. for 12-120 h.

33. The method according to any one of claims 14 to 25, characterized in that R represents substituted or unsubstituted alkenyl.

34. The method according to claim 33, characterized in that R is 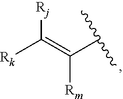
wherein Rⱼ, Rₖ, and Rₘ are any group.

35. The method according to claim 34, characterized in that R¹ and R² are each independently selected from any group, and the reaction scheme is as follows:

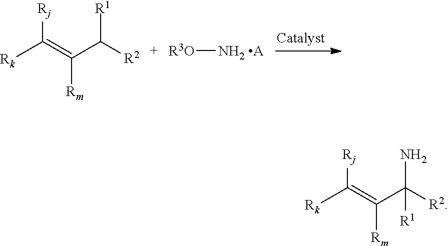

36. The method according to claim 35, characterized in that R¹ and R² are each independently selected from the group consisting of H and straight or branched C₁₋₁₀ alkyl; Rⱼ, Rₖ, and Rₘ are each independently selected from the group consisting of H and straight or branched C₁₋₁₀ alkyl.

37. The method according to claim 35, characterized in that the reaction conditions are: reacting in dioxane and water at 20-60° C. for 12-48 h, and preferably reacting at 25-40° C. for 12-24 h.

38. The method according to claim 33, characterized in that said R is 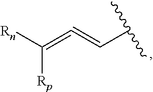
wherein Rₙ and Rₚ are any group.

39. The method according to claim 38, characterized in that R¹ and R² are each independently selected from any group, and the reaction scheme is as follows:

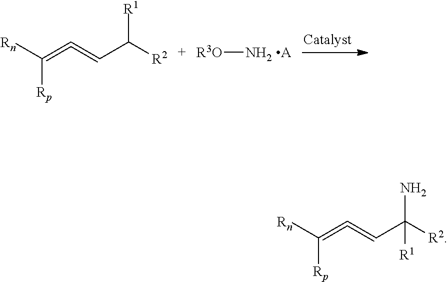

40. The method according to claim 39, characterized in that the reaction conditions are: reacting in a mixed solvent of dioxane and water at 20-60° C. for 12-48 h.

41. The method according to any one of claims 14 to 25, characterized in that said R is substituted or unsubstituted straight or branched alkyl, and R¹ and R² are each independently selected from any group;
the substituent is selected from any one or more of halogen, ester group, alkoxy, phenyl, and benzyl.

42. The method according to claim 41, characterized in that R is straight or branched C₁₋₁₈ alkyl, and R¹ and R² are each independently selected from the group consisting of H or straight or branched C₁₋₁₈ alkyl;
preferably, R is straight or branched C₁₋₁₀ alkyl, and R¹ and R² are each independently selected from the group consisting of H or straight C₁₋₁₀ alkyl.

43. The method according to any one of claims 14 to 25, characterized in that R and R¹ are linked to form a ring, and R² is H.

44. The method according to claim 43, characterized in that R and R¹ are linked to form a saturated ring, and the reaction scheme is as follows: 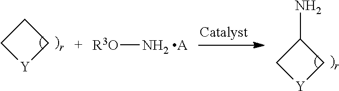
wherein, Y represents CHR″, r represents any integer from 0 to 12; R″ represents any group, and is preferably selected from the group consisting of H, methyl, ethyl, or benzyl.

45. The method according to claim 44, characterized in that the reaction conditions are: reacting in a mixed solvent of dioxane and water at 25-80° C. for 12-48 h.

46. The method according to any one of claims 14 to 25, characterized in that the structure of said compound 1 is selected from the group consisting of following structures:

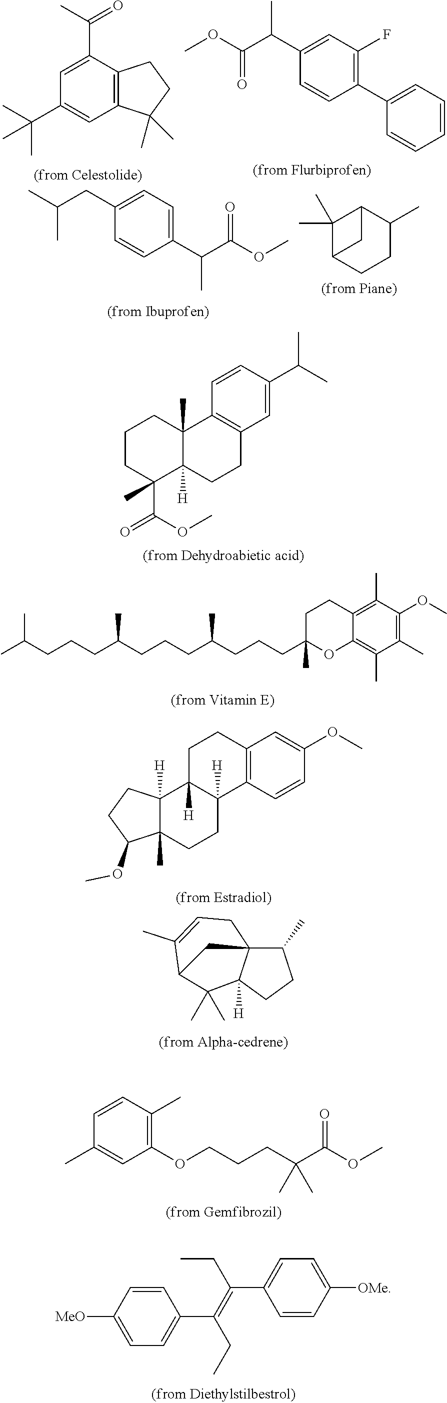

47. The method according to any one of claims 14 to 25, characterized in that R, R¹, and R² are linked to form a bridged ring.

48. The method according to claim 47, characterized in that compound 1 is selected from 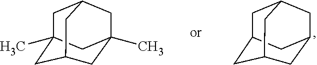
and the reaction scheme is as follows:

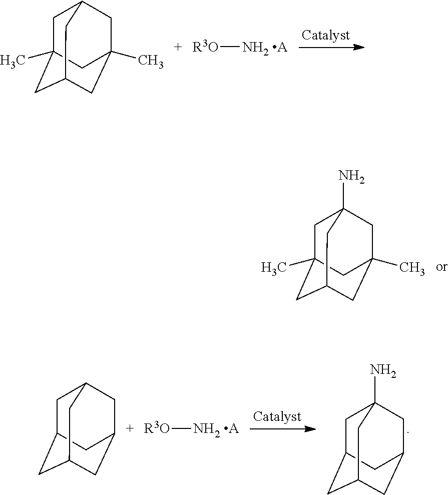

49. The method according to claim 48, characterized in that the reaction conditions are: reacting in a mixed solvent of dioxane and water at 20-80° C. for 12-72 h, and preferably reacting at 25° C. for 12 h.

50. The method according to any one of claims 14 to 25, characterized in that R represents substituted or unsubstituted alkynyl.

51. The method according to claim 50, characterized in that said R is 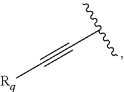
wherein R^{q} is any group.

52. The method according to claim 51, characterized in that R¹ and R² are each independently any group, and the reaction scheme is as follows:

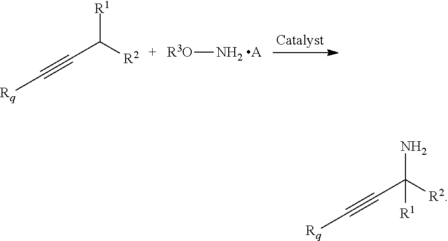

53. The method according to claim 52, characterized in that R¹ and R² are independently selected from the group consisting of H, phenyl, and straight or branched C₁₋₁₀ alkyl; R_{q} is selected from the group consisting of H, phenyl, and straight or branched C₁₋₁₀ alkyl.

54. The method according to claim 52, characterized in that the reaction conditions are: reacting in a mixed solvent of dioxane and water at 20-60° C. for 12-48 h.

55. The method according to any one of claims 14 to 25, characterized in that the structure of said compound 1 is selected from the group consisting of:

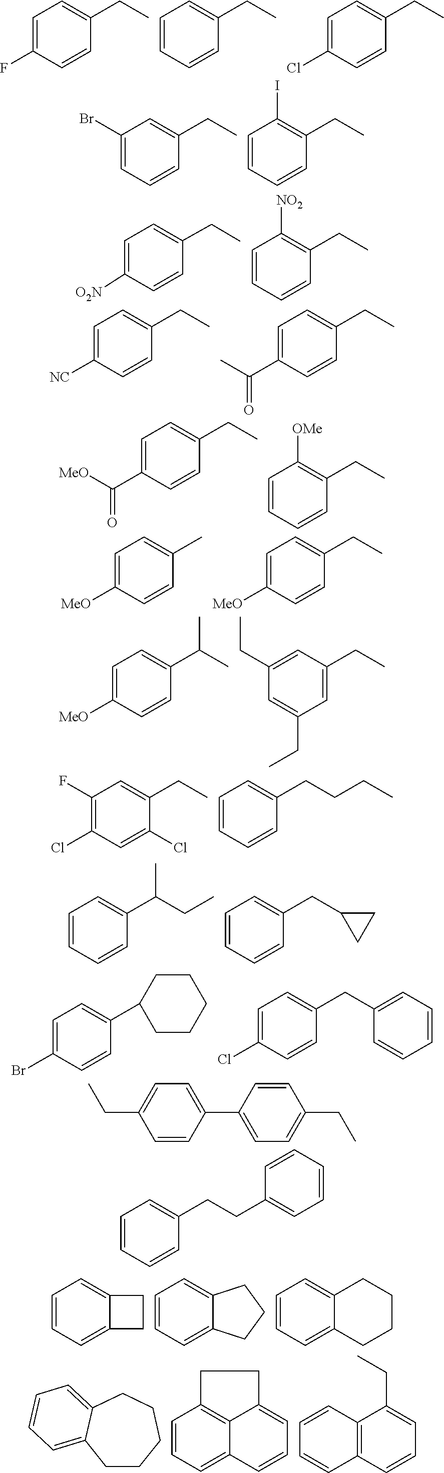

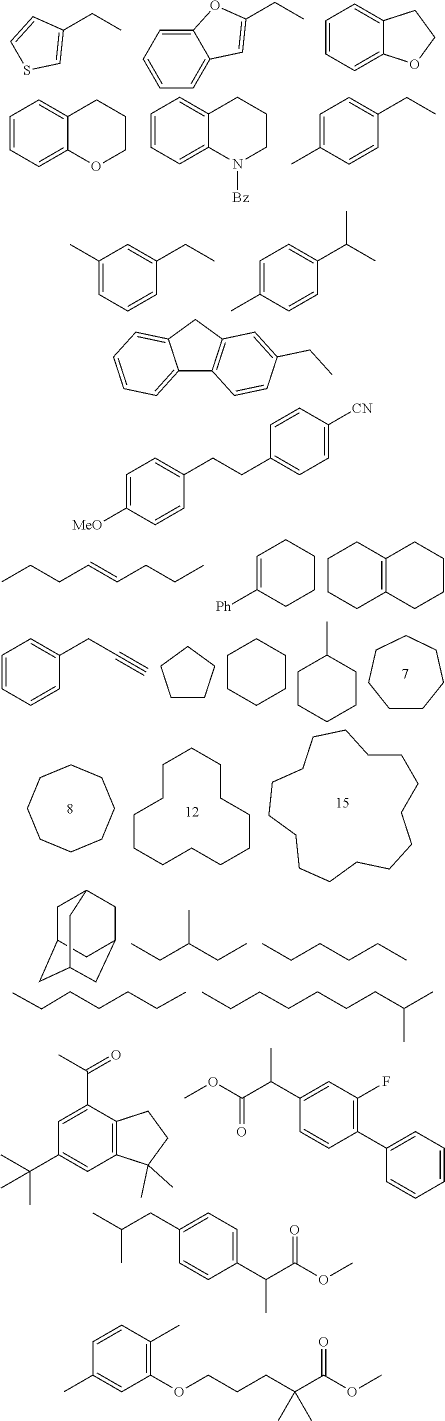

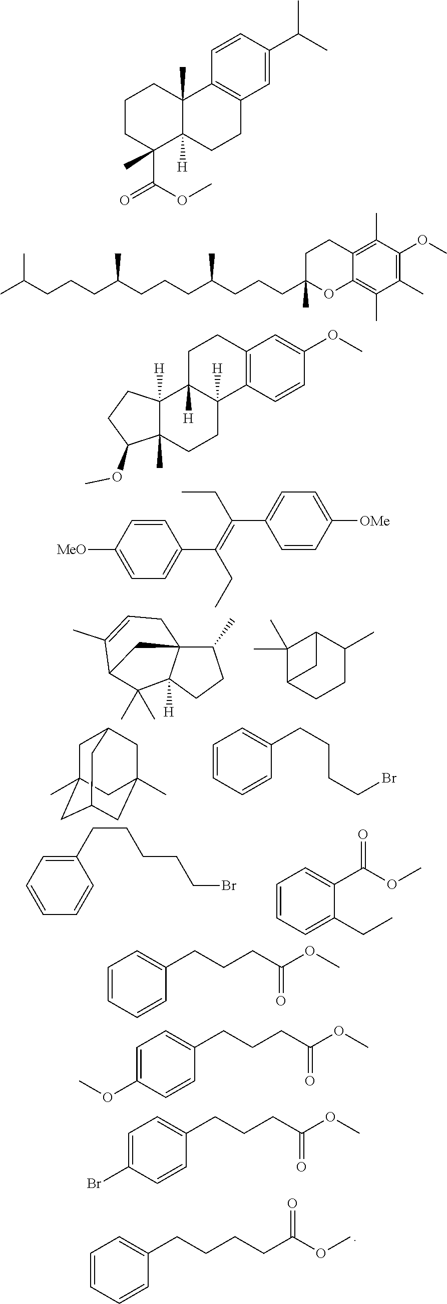

56. A method for synthesizing cyclic secondary amines or lactams, characterized in that the method comprises the step of cyclizing the primary amine compound prepared by the method described in claim 41 under the action of a base, to produce a cyclic secondary amine or lactam compound.

57. The method according to claim 56, characterized in that R represents substituted C₁₋₅ straight alkyl, and R¹ and R² are each independently selected from any group; the substituent is selected from the group consisting of halogen or ester group;
the reaction scheme is as follows: 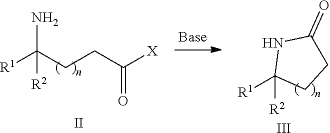
wherein, X is selected from the group consisting of halogen or alkoxy, and n is an integer ranging from 1 to 3.

58. The method according to claim 57, characterized in that R¹ is substituted or unsubstituted phenyl, R² is hydrogen, and n is 1 or 2; the substituent is selected from the group consisting of halogen or methyl ester group.
